(19) United States
(12) Patent Application Publication
(10) Pub. No.: US 2020/0138921 A1
(43) Pub. Date: May 7, 2020

(54) METHODS OF PREVENTING AND TREATING DISEASES CHARACTERIZED BY SYNAPTIC DYSFUNCTION AND NEURODEGENERATION INCLUDING ALZHEIMER'S DISEASE

(71) Applicant: The Trustees of Columbia University in the City of New York, New York, NY (US)

(72) Inventor: Roger Lefort, New York, NY (US)

(21) Appl. No.: 16/625,222

(22) PCT Filed: Jun. 25, 2018
(86) PCT No.: PCT/US18/39292

§ 371 Date: Dec. 20, 2019

Related U.S. Application Data
(60) Provisional application No. 62/524,282, filed on Jun. 23, 2017.
(60) Provisional application No. 62/624,258, filed on Jan. 31, 2018.

(51) Int. Cl. A61K 38/53; A61K 31/4745; A61P 25/28

(52) U.S. Cl. CPC A61K 38/53; A61P 25/28; C12Y 603/02019; A61K 31/4745

(57) ABSTRACT

This invention relates to methods and compositions for preventing and treating certain diseases or disorders characterized by synaptic dysfunction and neurodegeneration, including Alzheimer's disease by increasing the levels of a certain protein ubiquitin ligase Ube3A.

### CROSS REFERENCE TO RELATED APPLICATION

The present application claims priority to U.S. Patent Application Ser. No. 62/524,282 filed Jun. 23, 2017 and U.S. Patent Application Ser. No. 62/624,258 filed Jan. 31, 2018, both of which are hereby incorporated by reference in their entirety.

### FIELD OF THE INVENTION

This invention is in the field of preventing and treating certain diseases or disorders characterized by synaptic dysfunction and neurodegeneration, including Alzheimer's disease by increasing the levels of a certain protein ubiquitin ligase Ube3A, as well as in the field of screening for agents that prevent and treat these diseases.

### BACKGROUND OF THE INVENTION

Alzheimer's disease (“AD”) is a devastating neurodegenerative disease and the most common cause of dementia in the elderly. In 2015, approximately forty-four million people worldwide were estimated to suffer from AD or related dementia. To date, there are no effective treatments to cure or prevent the disease. The most common therapeutic strategy has been to directly target neurotic amyloid-beta (Aβ) peptide. However, this strategy has not proven successful. The catastrophic and costly failure of Aβ-centric strategies highlight the need for novel approaches to AD.

Alzheimer's disease, while classified as a neurodegenerative disease, is, at its core, a disease of synapses. Mounting evidence suggests that impairment of cognitive abilities typically seen in the earliest clinical phases are due to prominent synaptic alterations and synapse loss, particularly in the entorhinal cortex (EC) and the hippocampus, the principal areas affected in AD, and primarily not due to neuronal death (Scheff et al. 1990; Scheff et al. 1993; Scheff et al. 1996). While the precise molecular mechanism remains unclear, it is widely accepted that AD-associated synaptopathy is caused by elevated levels of soluble oligomeric β-amyloid (Aβ), which specifically targets synapses and disrupts various signaling molecules and pathways involved in synaptic function (Lacor et al. 2004; Lauren et al. 2009). A number of candidate molecules have been linked to AD-associated synaptic dysfunction.

Of particular interest, are members of the Rho-family of guanosine triphosphatases (GTPases), a subfamily of the Ras superfamily of GTPases, RhoA, Rac1 and Cdc42, which regulate synapses, more specifically dendritic spine morphology and function, by regulating the actin cytoskeleton, the main structural component of dendritic spines (Schaefer et al. 2014; Tashiro and Yuste 2008). RhoA favors the destabilization of dendritic spines, while Rac1 and Cdc42 promotes their stabilization and maturation (Woolfrey and Srivastava 2016). Given their critical role in synaptic function, aberrant Rho-GTPase signaling leads to widespread neuronal network dysfunction and has been proposed to play a key role in AD. RhoA subcellular mislocalization and altered levels has been reported both in human AD brains, and in the human amyloid precursor protein (hAPP) Tg2576 (Swedish mutation) AD mouse model (Hsiao et al. 1996; Huesa et al. 2010). Furthermore, the inventor recently demonstrated that synaptic dysfunction and synapse loss in cultured hippocampal neurons exposed to soluble oligomeric Aβ, and in the J20 hAPP AD mouse model (Mucke et al. 2000), are both preceded and dependent on increased RhoA activity (Pozueta et al. 2013).

Another important molecule implicated in AD-associated synaptic dysfunction is the activity regulated immediate-early gene (IEG) Arc/Arg3.1. Arc plays a critical role in synaptic plasticity and memory formation by regulating postsynaptic trafficking of AMPA-type glutamate receptors at excitatory synapses during long-term potentiation (LTP) consolidation and long-term depression (LTD) evocation (Korb and Finkbeiner 2011). Several lines of evidence have shown Arc levels to be altered in human AD brains, in various AD mouse models, and in cultured hippocampal neurons exposed to oligomeric Aβ (Perez-Cruz et al. 2011; Wu et al. 2011; Grinevich et al. 2009). How these two critical signaling molecules are dysregulated is currently unknown. However, they share one common regulatory molecule, the ubiquitin-protein ligase E3A, Ube3A/E6-AP (“Ube3A”) (Buiting et al. 2015).

Ube3A is best known for its causative role in the rare neurodevelopmental disorder, Angelman Syndrome (AS). AS is characterized by microcephaly, severe intellectual deficits, abnormal sleep patterns and hyperactivity, and is due in part to the loss of function of the imprinted UBE3A gene, located on chromosome 15q11.2-q13 (Buiting et al. 2106). Several potential Ube3A substrates have been identified, including ECT2, p53, p27, HR23A, Blk, and interestingly Arc and the RhoA-specific nuclear guanine nucleotide exchange factor (GEF) Ephexin-5, also known as ARHGEF15 (Huibregtse et al. 1992; Kuhnle et al. 2013; Kumar et al. 1999; Margolis et al. 2010; Mishra et al. 2009; Reiter et al. 2006; Oda et al. 1999). One interesting characteristic observed in AS mouse models include a significant reduction of dendritic spine density and length in neurons of the hippocampus and cortex, akin to AD mouse models (Kuhnle et al. 2013).

### SUMMARY OF THE INVENTION

The current invention is based upon the surprising discovery that restoring ubiquitin-protein ligase E3A, Ube3A/E6-AP (“Ube3A”) levels in the brain prevents synaptic dysfunction and elimination. Without being bound by any theory, Ube3A deficiency leads to the accumulation of two of its downstream targets, Arc and Ephexin-5, leading to synaptic dysfunction and elimination, respectively.

Thus, one embodiment of the present invention is a method of preventing and/or treating a disease characterized by synaptic dysfunction, comprising administering to a subject in need thereof a therapeutically effective amount of an agent which increases the levels of Ube3A. In one embodiment, the agent increases the levels of Ube3A in the brain.

Diseases that can prevented and/or treated by the methods of the invention include those characterized by synaptic dysfunction, so called synaptopathies, including but not limited to Alzheimer's disease (“AD”), Parkinson disease, Huntington's disease, and epilepsy. Diseases or conditions that cause neurodegeneration can also be prevented and/or treated by the methods of the invention.

The agent can be in many forms including but not limited to chemicals, pharmaceuticals, biologics, small organic molecules, antibodies, nucleic acids, peptides, and proteins. In one embodiment, the agent is a topoisomerase type I inhibitor. In some embodiments, the topoisomerase inhibitor type I is chosen from the group consisting of topotecan and irinotecan.

In some embodiments, the agent further comprises a pharmaceutically acceptable carrier and is part of a composition.

In some embodiments, the agent is a nucleic acid which encodes Ube3A, or the entire Ube3A gene, or a nucleic acid that is substantially homologous to the Ube3A gene, or a variant, mutant, fragment, homologue or derivative of the Ube3A gene.

Alternatively, the agent can be purified Ube3A protein combined with or in an appropriate carrier, ligand, conjugate, vector, lipid, carrier, adjuvant or diluent, such as an adeno-associated virus (AAV).

A further embodiment of the present invention is a kit comprising compositions and agents for practicing the invention.

A further embodiment of the present invention is a method and/or assay for screening and/or identifying an agent for the treatment and/or prevention of a disease characterized by synaptic dysfunction, including but not limited to AD.

A further embodiment of the present invention is a method and/or assay for screening and/or identifying a test agent for the prevention and/or treatment of a disease characterized by synaptic dysfunction and/or neurodegeneration, including but not limited to AD, comprising contacting or incubating a test agent with a nucleotide comprising Ube3A gene or a portion thereof, and detecting the expression of the nucleotide before and after contact or incubation with the test agent, wherein if the expression of the nucleotide is increased after the contact or incubation with the test agent, the test agent is identified as a therapeutic and/or preventative agent for a disease characterized by synaptic dysfunction and/or neurodegeneration.

A further embodiment of the present invention is a method and/or assay for screening and/or identifying a test agent for the prevention and/or treatment of a disease characterized by synaptic dysfunction, including but not limited to AD, comprising contacting or incubating a test agent with a gene construct comprising a nucleotide comprising the Ube3A gene or a portion thereof, and detecting the expression of the nucleotide in the gene construct before and after contacting or incubating the test agent with the gene construct, wherein if the expression of the gene is increased after contact with the test agent, the test agent is identified as a therapeutic and/or preventative agent for a disease characterized by synaptic dysfunction and/or neurodegeneration.

A further embodiment of the present invention is a method and/or assay for screening and/or identifying a test agent for the prevention and/or treatment of a disease characterized by synaptic dysfunction and/or neurodegeneration, including but not limited to AD, comprising transforming a host cell with a gene construct comprising a nucleotide comprising the Ube3A gene or a portion thereof, detecting the expression of the nucleotide in the host cell, contacting the test agent with the host cell, and detecting the expression of the nucleotide in the host cell after contact with the test agent or compound, wherein if the expression of the nucleotide is increased after contact with the test agent, the test agent is identified as a therapeutic and/or preventative agent for a disease characterized by synaptic dysfunction and/or neurodegeneration.

The expression of a nucleotide or gene can be determined using a measurable phenotype, either one that is native to the gene or one that is artificially linked, such as a reporter gene.

The invention also includes any agents identified by the screening methods described herein and methods of using the same for the prevention and/or treatment of a disease characterized by synaptic dysfunction and/or neurodegeneration.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1 shows that Tg2576 mice have behavioral deficits and dendritic spine abnormalities. FIG. 1A is a graph showing the results of Morris water maze (MWM) 7-day training showing latency (time in seconds) to find the platform in wild-type (WT) and Tg2576 mice. FIG. 1B is a graph showing the results of a Morris water maze (MWM) probe test showing the distribution of time spent (%) in each quadrant in the absence of platform (total time 60 sec). The diagram on the right shows the pool and the location of each quadrant. FIG. 1C is a quantification of hippocampal dendritic spines in WT and Tg2576 mice. The graphs represent mean values and standard deviation. Significance determined in FIGS. 1A and 1B by two-way ANOVA followed by Bonferroni Post Hoc multiple comparisons and in FIG. 1C by unpaired student's t-test.

FIG. 2 shows that Ube3A is decreased in Tg2576 mice hippocampus. FIG. 2A is a quantification of a Western blot of RhoA-GTP and RhoA (total) in the hippocampus of wild-type (WT) and Tg2576. FIG. 2B shows a representative Western blot of WT and Tg2576 of Ube3A, Arc, Ephexin-5 and GAPDH (as house-keeping protein). FIG. 2C is a graph of the quantification of relative protein levels of Ephexin-5. FIG. 2D is a graph of the quantification of relative protein levels of Ube3A. FIG. 2E is a graph of the quantification of the relative protein levels of Arc. Graphs represent mean values and standard deviation. Significance determined by unpaired student's t test.

FIG. 3 shows that Ube3A is decreased in rat primary hippocampal neurons in presence of oAβ. FIG. 3A is a graph of the quantification of spine density in vehicle treated (control) and oAβ treated neurons when labelled with βIII-tubulin and counterstained with DAPI. FIG. 3B is a representative Western blot of Rho-GTP (Active) and RhoA (Total) in control (vehicle treated) and oAβ treated neurons. FIG. 3C is a graph of the quantification of the Western blot in FIG. 3B. FIG. 3D is a representative Western blot of Ube3A and surface-expressed EphB2 relative to total EphB2 control and oAβ treated neurons. FIG. 3E is a graph of the quantification of Ube3A relative to total EphB2. FIG. 3F is a graph of the quantification of surface-expressed EphB2 relative to total EphB2. Graphs represent mean values and standard deviation. Significance determined by unpaired student's t-test.

FIG. 4 shows oAβ induces c-Abl phosphorylation and Ube3A destabilization in rat primary hippocampal neurons. FIG. 4A is a quantification of Western blots of phosphorylated c-Abl in vehicle treated (control) and DPH treated hippocampal neurons. FIG. 4B is a quantification of Western blots of phosphorylated Ube3A in vehicle treated (control) and DPH treated hippocampal neurons. FIG. 4C is a quantification of Western blots of phosphorylated c-Abl in vehicle treated (control), oAβ treated, and oAβ with STI treated (STI-571; c-Abl inhibitor) hippocampal neurons. FIG. 4D is a quantification from Western blots of phosphorylated Ube3A in vehicle treated (control), oAβ treated, and oAβ with STI treated hippocampal neurons. Graphs represent mean values and standard deviation. Significance determined by unpaired student's t-test in FIGS. 4A and 4B, and one-way ANOVA followed by Tukey Post Hoc multiple comparisons in FIGS. 4C and 4D.

FIG. 5 shows that oAβ increases the degradation of Ube3A. FIG. 5A is a graph showing the quantification of Western blots of the levels of Ube3A and GAPDH (as house-keeping protein) of B35 cells treated with cycloheximide (CHX) or CHX and oAβ for 4, 8 and 16 hours. FIG. 5B is a graph showing the quantification of Western blots of the levels of Ube3A and GAPDH (as house-keeping protein) of B35 cells treated oAβ with or without STI-571, a c-Abl inhibitor and vehicle treated (control). Graphs represent mean values and standard deviation. Significance determined by one-way ANOVA followed by Tukey Post Hoc multiple comparisons.

FIG. 6 shows that oAβ dysregulates Ube3A downstream proteins in rat hippocampal neurons. FIG. 6A is a representative Western blot of Ephexin-5, Arc, p53, and surface GluR1 of neurons treated with and without oAβ. FIG. 6B is a graph of the quantification of Ephexin-5 in neurons treated with and without oAβ. FIG. 6C is a graph of the quantification of Arc in neurons treated with and without oAβ. FIG. 6D is a graph of the quantification of p53 in neurons treated with and without oAβ. FIG. 6E is a graph of the quantification of surface GluR1 in neurons treated with and without oAβ. FIG. 6F is a graph showing the quantification of GluR1 fluorescence intensity of neurons treated with and without oAβ. Graphs represent mean values and standard deviation. Significance determined by unpaired student's t-test.

FIG. 7 shows that oAβ-induced RhoA activation is Ephexin-5 dependent. FIG. 7A is a graph of the quantification of Western blots of Ephexin-5 and GAPDH (as house-keeping protein) in rat hippocampal neurons treated with and without oAβ. FIG. 7B is a representative Western blot of the input (Total RhoA) and pull-down fraction (Active RhoA) of control, oAβ, and oAβ and siEphexin-5 treated neurons. FIG. 7C is a graph of the quantification of Western blots of RhoA-GTP relative to total RhoA of control, oAβ, and oAβ and siEphexin-5 treated neurons. FIG. 7D is a graph of the quantification of spine density of control, oAβ, and oAβ and siEphexin-5 treated neurons. Graphs represent mean values and standard deviation. Significance determined by unpaired student's t-test in FIG. 7A, and one-way ANOVA followed by Tuckey Post Hoc multiple comparisons in FIGS. 7C and 7D.

FIG. 8 shows that restoring Ube3A protects against oAβ-induced synaptotoxicity. FIG. 8A is a schematic of a timeline of the treatment protocol. FIG. 8B is a representative Western blot of DD-Ube3A, endogenous Ube3A, Ephexin-5, Arc, and surface and total GluR1 of neurons treated as shown in FIG. 8A. FIG. 8C is a graph of the protein quantification of Ephexin-5 from the neurons in the Western blot of FIG. 8B. FIG. 8D is a graph of the protein quantification of PSD 95 from the neurons in the Western blot of FIG. 8B. FIG. 8E is a graph of the protein quantification of Arc from the neurons in the Western blot of FIG. 8B. FIG. 8F is a graph of the protein quantification of surface GluR1 from the neurons in the Western blot of FIG. 8B. FIG. 8G is a graph of spine density quantification of neurons treated as described in FIG. 8A. Graphs represent mean values and standard deviation. Significance determined by one-way ANOVA followed by Tuckey Post Hoc multiple comparisons.

FIG. 9 shows topotecan treatment increased Ube3A levels, concomitant with decreased Ephexin-5 levels. FIG. 9A is a graph of the quantification of Western blots of Ube3A protein in vehicle and topotecan treated neurons at 24 and 72 hours (p=0.002, ANOVA, N=3). FIG. 9B is a graph of the quantification of Western blots of Ephexin-5 protein in vehicle and topotecan treated neurons (*p=0.04, ***p=0.0005, ANOVA, N=3). FIG. 9C is a graph of the percent of live cells relative to controls of vehicle and topotecan treated neurons.

FIG. 10 shows that shows topotecan treatment reversed oAβ-induced RhoA activation. FIG. 10A is a graph of the quantification of Western blots of Ube3A protein in vehicle and topotecan treated neurons also treated with oAβ (p=0.008, ANOVA). FIG. 10B is a graph of the quantification of Western blots of RhoA-GTP, Rac1-GTP, and Cdc42-GTP of control, oAβ, and oAβ and topotecan treated neurons (**p=0.003, **p=0.0008). Graphs represent mean values and standard deviation.

FIG. 11 shows that topotecan treatment reversed oAβ-induced loss of spine density. FIG. 11A is a graph of the quantification of spine density of control, oAβ, oAβ and topotecan treated, and topotecan only treated neurons. FIG. 11B is a graph of the quantification of Western blots of PSD95 levels relative to control of control, oAβ, oAβ and topotecan treated, and topotecan only treated neurons (*p=0.029). FIG. 11C is a graph of the quantification of Western blots of NMDARI levels relative to control of control, oAβ, oAβ and topotecan treated, and topotecan only treated neurons (*p=0.041). Graphs represent mean values and standard deviation.

FIG. 12 shows a single dose of topotecan in wild-type mice resulted in increased Ube3A protein as well as enhanced memory, long-term potentiation (LTP), and increased dendritic spine density. FIG. 12A is a graph of the quantification of Western blots of Ube3A protein in control (untreated) and topotecan treated wild-type mice (*p=0.05, ANOVA, N=8). FIG. 12B is a graph showing the results of Morris water maze (MWM) 7-day training showing latency (time; sec) to find the platform in control (untreated) (left hand bars) and topotecan treated (right hand bars) wild type mice (*p<0.05, ANOVA, N=15 control, N=19 topotecan treated). FIG. 12C is a graph showing results of LTP (fEPSP slope versus time) in vehicle treated mice (N=10 bottom scan) and topotecan treated wild-type mice (N=11 (top scan)) (8 mice per group, *p=0.017, ANOVA). FIG. 12D is a graph showing the dendritic spine density in the hippocampus of vehicle treated and topotecan treated wild type mice (*p<0.05, t-test, N=12 slices, 8 mice per group).

FIG. 13 shows a single dose of topotecan in AD mice reversed the AD phenotype and resulted in an increase in Ube3A protein. FIG. 13A is a graph of the quantification of Western blots of Ube3A protein in wild-type, control J20 (untreated), and topotecan treated J20 mice (*p=0.014,**p=0.002, ANOVA). FIG. 13B is a graph of latency to platform in seconds over the days of the Morris water maze (MWM) testing of PBS wild-type treated mice (N=17), PBS treated J20 mice (N=16), and topotecan treated J20 mice (N=12). FIG. 13C is a graph showing the results of Morris water maze (MWM) time in target quadrant in seconds of PBS wild-type treated mice, PBS treated J20 mice, and topotecan treated J20 mice (*p<0.05, **p<0.01, ANOVA). FIG. 13D are graphs showing the speed in cm/s and time to platform of PBS wild-type treated mice, PBS treated J20 mice, and topotecan treated J20 mice. FIG. 13E are graphs showing the results of LTP (fEPSP versus time) in PBS wild type treated mice (N=10), PBS treated J20 mice (N=9), and topotecan treated J20 mice (N=11), and residual potentiation of each mice (p<0.05). FIG. 13F is a graph showing the dendritic spine density in the hippocampus of PBS wild-type treated mice (N=9), PBS treated J20 mice (N=10), and topotecan treated J20 mice (N=12) (*p<0.05, **p<0.01, t-test).

FIG. 14 shows that topotecan treatment does not affect Aβ but does decrease phospho-tau. FIG. 14A is representative images at 14 months of hippocampal slices stained with DAPI, Aβ (6E10) and GFAP antibodies. FIG. 14B is a graph of the quantification of a Western blot of phospho-tau in control J20 (untreated) and topotecan treated J20 mice (**p<0.004, ANOVA).

### DETAILED DESCRIPTION OF THE INVENTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “subject” as used in this application means an animal with an immune system such as avians and mammals Mammals include canines, felines, rodents, bovine, equines, porcines, ovines, and primates. Avians include, but are not limited to, fowls, songbirds, and raptors. Thus, the invention can be used in veterinary medicine, e.g., to treat companion animals, farm animals, laboratory animals in zoological parks, and animals in the wild. The invention is particularly desirable for human medical applications.

The term “patient” as used in this application means a human subject. In some embodiments of the present invention, the “patient” is suffering with Alzheimer's disease. In some embodiments, the patient is suffering from another disease or condition that causes neurodegeneration and/or synaptic dysfunction.

The terms “Alzheimer's disease” and “AD” will be used interchangeable and is a disease in which there is mild cognitive impairment and the presence of amyloid plaques comprising Aβ.

The terms “treat”, “treatment”, and the like refer to a means to slow down, relieve, ameliorate or alleviate at least one of the symptoms of the disease, or reverse the disease after its onset.

The terms “prevent”, “prevention”, and the like refer to acting prior to overt disease onset, to prevent the disease from developing or minimize the extent of the disease or slow its course of development.

The term “in need thereof” would be a subject known or suspected of having or being at risk of Alzheimer's disease, or a disease or condition that causes neurodegeneration and/or a synaptic dysfunction. In the case of AD, the subject may be suffering from cognitive impairment ranging from mild to severe, or with pre-dementia in the prodromal phase

A subject in need of treatment would be one that has already developed the disease. A subject in need of prevention would be one with risk factors of the disease.

The term “agent” as used herein means a substance that produces or is capable of producing an effect and would include, but is not limited to, chemicals, pharmaceuticals, biologics, small organic molecules, antibodies, nucleic acids, peptides, and proteins.

The phrase “therapeutically effective amount” is used herein to mean an amount sufficient to cause an improvement in a clinically significant condition in the subject, or delays or minimizes or mitigates one or more symptoms associated with the disease, or results in a desired beneficial change of physiology in the subject.

The terms “screen” and “screening” and the like as used herein means to test an agent to determine if it has a particular action or efficacy.

The terms “identification”, “identify”, “identifying” and the like as used herein means to recognize an agent as being effective for a particular use.

As used herein, the term “isolated” and the like means that the referenced material is free of components found in the natural environment in which the material is normally found. In particular, isolated biological material is free of cellular components. In the case of nucleic acid molecules, an isolated nucleic acid includes a PCR product, an isolated mRNA, a cDNA, an isolated genomic DNA, or a restriction fragment. In another embodiment, an isolated nucleic acid is preferably excised from the chromosome in which it may be found. Isolated nucleic acid molecules can be inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated material may be, but need not be, purified.

The term “purified” and the like as used herein refers to material that has been isolated under conditions that reduce or eliminate unrelated materials, i.e., contaminants. For example, a purified protein is preferably substantially free of other proteins or nucleic acids with which it is associated in a cell and a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include, but are not limited to, plasmids, phages, and viruses.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily be introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts, and many appropriate host cells, are known to those skilled in the relevant art. Recombinant cloning vectors will often include one or more replication systems for cloning or expression, one or more markers for selection in the host, e.g. antibiotic resistance, and one or more expression cassettes.

The term “host cell” means any cell of any organism that is selected, modified, transformed, grown, used or manipulated in any way, for the production of a substance by the cell, for example, the expression by the cell of a gene, a DNA or RNA sequence, a protein or an enzyme. Host cells can further be used for screening or other assays, as described herein.

A “polynucleotide” or “nucleotide sequence” is a series of nucleotide bases (also called “nucleotides”) in a nucleic acid, such as DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide. This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates) and with charged linkages (e.g., phosphorothioates, phosphorodithioates). Polynucleotides may contain one or more additional covalently linked moieties, such as, for example, proteins (e.g., nucleases, toxins, antibodies, signal peptides, poly-L-lysine), intercalators (e.g., acridine, psoralen), chelators (e.g., metals, radioactive metals, iron, oxidative metals), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

The terms “percent (%) sequence similarity”, “percent (%) sequence identity”, and the like, generally refer to the degree of identity or correspondence between different nucleotide sequences of nucleic acid molecules or amino acid sequences of proteins that may or may not share a common evolutionary origin. Sequence identity can be determined using any of a number of publicly available sequence comparison algorithms, such as BLAST, FASTA, DNA Strider, or GCG (Genetics Computer Group, Program Manual for the GCG Package, Version 7, Madison, Wis.).

The terms “substantially homologous” or “substantially similar” means when at least about 80%, and most preferably at least about 90 or 95%, 96%, 97%, 98%, or 99% of the nucleotides match over the defined length of the DNA sequences, as determined by sequence comparison algorithms, such as BLAST, FASTA, and DNA Strider. An example of such a sequence is an allelic or species variant of the specific genes of the invention. Sequences that are substantially homologous can be identified by comparing the sequences using standard software available in sequence data banks, or in a Southern hybridization experiment under, for example, stringent conditions as defined for that particular system.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

### Dysfunction of the Ubiquitin Ligase E3A Ube3A/E6-AP Contributes to Synaptic Pathology in Alzheimer's Disease

As discussed above, while widespread synaptic dysfunction and the loss of synapses are invariable features of AD, the exact mechanism(s) driving these changes remains unclear. Accumulating evidence suggests that aberrant signaling by two key synaptic proteins may play an important role in AD-associated synaptopathy, namely Arc/Arg3.1 and Ephexin-5, whose levels are anomalously upregulated in the brain of AD patients, and in various AD mouse models. The exact mechanism(s) leading to the upregulation of these two proteins is not fully understood. However, several prior studies suggest that oligomeric Aβ may be at least partially responsible, as it can induce their rapid expression in neurons. See Lacor et al. 2004; Perez-Cruz et al. 2011; Wu et al. 2011; Grinevich et al. 2009; Margolis et al. 2010; Sell et al. 2017.

As shown herein the degradation of both Arc and Ephexin-5 may also be impaired in AD, perhaps contributing to their sustained expression in neurons exposed to oligomeric Aβ. It is shown herein for the first time that the Angelman Syndrome (AS)-associated protein, Ube3A, a key E3 ligase regulating the degradation of Arc and Ephexin-5, was significantly downregulated in cognitively impaired AD transgenic mouse brains, and in cultured primary rat hippocampal neurons treated with oligomeric Aβ, suggesting that Ube3A is a key player in AD associated synaptic dysfunction (Examples 2 and 3). Consistent with this idea, the observed depletion of Ube3A paralleled the significant oAβ-induced increase in both Arc and Ephexin-5, which as previous studies have shown, leads to decreased surface-expressed GluR1 (due to increased Arc expression (Chowdhury et al. 2006)), and loss of dendritic spine density (through increased Ephexin-5 and RhoA activity) (Margolis et al. 2010; Sell et al. 2107)). See Examples 2 and 5. Interestingly, the data set forth herein indicated that the decrease in Ube3A in response to oAβ exposure appears to be specific to neurons, as Ube3A levels were conversely increased in cultured primary rat astrocytes, suggesting a differential response to oAβ between neurons and astrocytes (Example 3). Indeed, prior studies have shown that astrocytes, unlike neurons are resistant to oAβ cytotoxicity, possibly due to the activation of distinct signaling pathways (Sell et al. 2017).

The consequences of loss-of-function of Ube3A have been well documented in the context of Angelman syndrome, and there is growing evidence that gain-of-function of Ube3A may also play a role in Autism Spectrum Disorder (Yi et al. 2017). The phenotypic similarities between AS and other neurodevelopmental disorders, and now with AD, can probably be explained by the significant overlap in downstream signaling pathways, which suggest that therapeutic strategies developed for one disorder may also be applicable to others, especially to correct like-phenotypes. More importantly, the results herein show Ube3A as a prime target for AD therapeutics.

### Restoring Ube3A in Both Neurons and Mice Abrogates the Synaptic Pathology of AD and Increases Memory

Most importantly shown herein, the restoration of Ube3A in oAβ-treated neurons, was sufficient to completely blocked the induction of Arc and Ephexin-5, while also abrogating the loss of surface expressed GluR1, and dendritic spine density, confirming the contribution of Ube3A to these processes (Example 6). This restoration was done using a transgene encoding Ube3A.

Similar results were found when a pharmacological agent was used to restore Ube3A levels. Topoisomerase type I inhibitors increase neuronal Ube3A by unsilencing the paternal allele. Topotecan and irinotecan, both FDA approved chemotherapeutic agents, are topoisomerase type I inhibitors. Topotecan was administered to both neurons (Examples 7-9) and wild-type mice (Example 10). In both cases, Ube3A protein was increased. The treatment of the neurons also decreased Ephexin-5 with no effect on cell viability. Topotecan treated mice had increased memory and a significant increase in dendritic spine density. Additionally, the effects of one injection of a low dose of topotecan persisted up to five months suggesting a permanent unsilencing of the allele and there was no toxicity seen in the treated mice for nine months.

More remarkably, a single low dose of topotecan reversed the Alzheimer's disease phenotype of an AD mouse. The topotecan treated mice had increased Ube3A levels, increased memory, and a significant increase in dendritic spine density similar to the wild-type mice. Moreover, the effects of the treatment lasted four months (Example 11).

The results set forth herein show that Ube3A is a valuable target for the treatment and prevention of AD and that agents that restore Ube3A function can be therapeutic agents for AD and other diseases characterized by synaptic dysfunction and neurodegeneration.

Methods and Compositions for the Prevention and Treatment of Diseases Characterized by Synaptic Dysregulation and Neurodegeneration including Alzheimer's Disease

Methods of the current invention for preventing and treating diseases characterized by synaptic dysfunction as well as neurodegeneration including but not limited to Alzheimer's disease include the administration of a therapeutically effective amount of an agent which increases the level of the protein ubiquitin ligase E3A Ube3A/E6-AP or Ube3A.

Agents that can be used in this method include but are not limited to agents for increasing the expression of the gene encoding Ube3A, and include nucleic acids which encode the Ube3A protein, or the entire Ube3A gene, or a nucleic acid that is substantially homologous to the Ube3A gene, or a variant, mutant, fragment, homologue or derivative of the Ube3A gene that produces a protein that maintains or increases its function.

In an embodiment, the variant of the nucleic acid encoding Ube3A has at least 81% sequence identity with the sequence of the nucleotide of which it is a variant. Thus, preferably, the variant of the nucleotide has at least 81%, 82%, 83%, 84%, 85%, 86%, 87%, 88%, 89%, 90%, 91%, 92%, 93%, 94%, 95%, 96%, 97%, 98% or 99% sequence identity with the sequence of the Ube3A nucleotide.

The sequences for the Ube3A gene can be found on the National Center for Biotechnology Database and can be used to manufacture variants, mutants, fragments, homologues and derivatives which maintain or have increased function.

DNA or other nucleic acids such as mRNA can also be used in the method.

Methods for delivery include receptor mediated endocytosis where the nucleic acid is coupled to a targeting molecule that can bind to a specific cell surface receptor, inducing endocytosis and transfer of the nucleic acid into cells. Coupling is normally achieved by covalently linking poly-lysine to the receptor molecule and then arranging for (reversible) binding of the negatively charged DNA or RNA to the positively charged poly-lysine component. Another approach utilizes the transferrin receptor or folate receptor which is expressed in many cell types.

Another method to administer the nucleic acid to the proper tissue is direct injection/particle bombardment, where the nucleic acid is to be injected directly with a syringe and needle into a specific tissue, such as muscle, skin or tumor, and can be delivered by administration including intravenous, intradermal, and subcutaneous injection.

An alternative direct injection approach uses particle bombardment (‘gene gun’) techniques: nucleic acid is coated on to metal pellets and fired from a special gun into cells. Successful gene transfer into a number of different tissues has been obtained using this approach. Such direct injection techniques are simple and comparatively safe.

Another method for delivery of nucleic acid to the proper tissue or cell is by using adeno-associated viruses (AAV). Nucleic acid is delivered in these viral vectors is continually expressed, replacing the expression of the DNA or RNA that is not expressed in the subject. Also, AAV have different serotypes allowing for tissue-specific delivery due to the natural tropism toward different organs of each individual AAV serotype as well as the different cellular receptors with which each AAV serotype interacts. The use of tissue-specific promoters for expression allows for further specificity in addition to the AAV serotype.

Other mammalian virus vectors that can be used to deliver the DNA or RNA include oncoretroviral vectors, adenovirus vectors, Herpes simplex virus vectors, and lentiviruses.

While it would be understood that any agent or agents that increase or upregulate the expression of Ube3A, would also most likely increase the Ube3A protein, alternatively, an agent or agents that directly increase or promote the activation, amount and/or activity of the proteins can be used in the methods.

Alternatively, administering the proteins can be used in the methods. This includes the administration of a polypeptide, or a variant thereof having at least 80% sequence identity with the Ube3A polypeptides.

In an embodiment, the variant of the polypeptide has at least 81% sequence identity with the sequence of the polypeptide of which it is a variant. Thus, preferably, the variant of the polypeptide has at least 81%, 82%, 83%, 84%, 85%, 86%, 87%, 88%, 89%, 90%, 91%, 92%, 93%, 94%, 95%, 96%, 97%, 98% or 99% sequence identity with the sequence of the Ube3A polypeptide. Such variants may be made, for example, using the methods of recombinant DNA technology, protein engineering and site-directed mutagenesis, which are well known in the art, and discussed in more detail below.

The percent sequence identity between two polypeptides may be determined using suitable computer programs.

Biologically active fragments (also referred to as biologically active peptides) or variants include any fragments or variants of a protein that retain an activity of the protein.

Polypeptides may be prepared using an in vivo or in vitro expression system. Preferably, an expression system is used that provides the polypeptides in a form that is suitable for pharmaceutical use, and such expression systems are known to the skilled person. As is clear to the skilled person, polypeptides of the invention suitable for pharmaceutical use can be prepared using techniques for peptide synthesis.

A nucleic acid molecule encoding, for example, the protein or variants thereof, may be used to transform a host cell or host organism for expression of the desired polypeptide. Suitable hosts and host cells are known in the art and may be any suitable fungal, prokaryotic or eukaryotic cell or cell line or organism, for example: bacterial strains, including gram-negative strains such as Escherichia coli and gram-positive strains such as Bacillus subtilis or of Bacillus brevis; yeast cells, including Saccharomyces cerevisiae; or Schizosaccharomyces pombe; amphibian cells such as Xenopus oocytes; insect-derived cells, such SF9, Sf21, Schneider and Kc cells; plant cells, for example tobacco plants; or mammalian cells or cell lines, CHO-cells, BHK-cells (for example BHK-21 cells) and human cells or cell lines such as HeLa, as well as all other hosts or host cells that are known and can be used for the expression and production of polypeptides.

The polypeptide or variants thereof, may be made by chemical synthesis, again using methods well known in the art for many years. In certain embodiments, polypeptides for administration to a patient may be in the form of a fusion molecule in which the polypeptide is attached to a fusion partner to form a fusion protein. Many different types of fusion partners are known in the art. One skilled in the art can select a suitable fusion partner according to the intended use of the fusion protein. Examples of fusion partners include polymers, polypeptides, lipophilic moieties, and succinyl groups. Certain useful protein fusion partners include serum albumin and an antibody Fc domain, and certain useful polymer fusion partners include, but are not limited to, polyethylene glycol, including polyethylene glycols having branched and/or linear chains. In certain embodiments, the polypeptide may be PEGylated, or may comprise a fusion protein with an Fc fragment.

In an embodiment, the polypeptide may be fused to or may comprise additional amino acids in a sequence that facilitates entry into cells (i.e. a cell-penetrating peptide). Thus, for example, the Ube3A protein or variant thereof or a polypeptide may further comprise the sequence of a cell-penetrating peptide (also known as a protein transduction domain) that facilitates entry into cells. As is well known in the art, cell-penetrating peptides are generally short peptides of up to 30 residues having a net positive charge and act in a receptor-independent and energy-independent manner.

Additionally or alternatively, the polypeptide may be fused to or may comprise additional amino acids in a sequence that facilitates entry into the nucleus (i.e., a nuclear localization sequence (NLS), aka nuclear localization domain (NLD)). Thus, for example, the Ube3A protein or variant thereof may further comprise the sequence of an NLS that facilitates entry into the nucleus. NLS includes any polypeptide sequence that, when fused to a target polypeptide, is capable of targeting it to the nucleus. Typically, the NLS is one that is not under any external regulation (e.g. calcineurin regulation) but which permanently translocates a target polypeptide to the nucleus.

It is appreciated that the sequence of the cell-penetrating peptide and/or the NLS may be adjacent to the sequence of the protein or variant, or these sequences may be separated by one or more amino acids residues, such as glycine residues, acting as a spacer.

Therapeutic proteins produced as an Fc-chimera are known in the art. For example, Etanercept, the extracellular domain of TNFR2 combined with an Fc fragment, is a therapeutic polypeptide used to treat autoimmune diseases, such as rheumatoid arthritis.

Other protein modifications to stabilize a polypeptide, for example to prevent degradation, as are well known in the art may also be employed. Specific amino acids may be modified to reduce cleavage of the polypeptide in vivo. Typically, N- or C-terminal regions are modified to reduce protease activity on the polypeptide. A stabilizing modification is any modification capable of stabilizing a protein, enhancing the in vitro half-life of a protein, enhancing circulatory half-life of a protein and/or reducing proteolytic degradation of a protein. For example, polypeptides may be linked to the serum albumin or a derivative of albumin. Methods for linking polypeptides to albumin or albumin derivatives are well known in the art.

The fusion partner may be attached, either covalently or non-covalently, to the amino-terminus or the carboxy-terminus of the polypeptide. The attachment may also occur at a location within the polypeptide other than the amino-terminus or the carboxy-terminus, for example, through an amino acid side chain (such as, for example, the side chain of cysteine, lysine, histidine, serine, or threonine).

One method for delivery of the protein to the proper tissue or cell is by using vectors, such as adeno-associated viruses (AAV). AAV have different serotypes allowing for tissue-specific delivery due to the natural tropism toward different organs of each individual AAV serotype as well as the different cellular receptors with which each AAV serotype interacts.

Delivery vehicles such as liposomes, nanocapsules, microparticles, microspheres, lipid particles, vesicles, and the like, may be used for the introduction of the compositions of the present invention into suitable host cells. In particular, vector delivered transgenes or proteins may be formulated for delivery either encapsulated in a lipid particle, a liposome, a vesicle, a nanosphere, or a nanoparticle or the like.

The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868; and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 tim. Sonication of MLVs results in the formation of small unilamellar vesicles (SUVs) with diameters in the range of 200 to 500 Å, containing an aqueous solution in the core.

Alternatively, nanocapsule formulations may be used. Nanocapsules can generally entrap substances in a stable and reproducible way.

Nanoparticles are a colloidal carrier system that has been shown to improve the efficacy of an encapsulated drug by prolonging the serum half-life. Polyalkylcyanoacrylates (PACAs) nanoparticles are a polymer colloidal drug delivery system that is in clinical development (described, for example, by Stella et al. (2000) J. Pharm. Sci., 89: 1452-1464; Brigger et al. (2001) Int. J. Pharm 214: 37-42; Calvo et al. (2001) Pharm. Res. 18: 1157-1166; and Li et al. (2001) Biol. Pharm. Bull. 24: 662-665). Biodegradable poly(hydroxyl acids), such as the copolymers of poly(lactic acid) (PLA) and poly(lactic-co-glycolide) (PLGA) are being extensively used in biomedical applications and have received FDA approval for certain clinical applications. In addition, nanoparticles have many desirable carrier features including (i) that the agent to be encapsulated comprises a reasonably high weight fraction (loading) of the total carrier system; (ii) that the amount of agent used in the first step of the encapsulation process is incorporated into the final carrier (entrapment efficiency) at a reasonably high level; (iii) that the carrier has the ability to be freeze-dried and reconstituted in solution without aggregation; (iv) that the carrier be biodegradable; (v) that the carrier system be of small size; and (vi) that the carrier enhances the particles persistence. Nanoparticles may be synthesized using virtually any biodegradable shell known in the art. Such polymers are biocompatible and biodegradable and are subject to modifications that desirably increase the photochemical efficacy and circulation lifetime of the nanoparticle. In one embodiment, the polymer is modified with a terminal carboxylic acid group (COOH) that increases the negative charge of the particle and thus limits the interaction with negatively charged nucleic acids. Nanoparticles may also be modified with polyethylene glycol (PEG), which also increases the half-life and stability of the particles in circulation. Alternatively, the COOH group may be converted to an N-hydroxysuccinimide (NHS) ester for covalent conjugation to amine-modified compounds.

The present invention also encompasses the administration of pharmacological agents, more specifically topoisomerase type I inhibitors and more specifically topotecan and irinotecan.

All of the agents discussed herein can be in the form of pharmaceutical compositions.

Most preferred methods of administration of the agents and compositions for use in the methods of the invention are oral, intrathecal, nasal, and parental including intravenous. The pharmacological agent must be in the appropriate form for administration of choice.

Such pharmaceutical compositions comprising one or more pharmacological agents for administration may comprise a therapeutically effective amount of the pharmacological agent and a pharmaceutically acceptable carrier.

The phrase “pharmaceutically acceptable” as used herein refers to molecular entities and compositions that are physiologically tolerable and do not typically produce an allergic or similar untoward reaction, such as gastric upset, dizziness and the like, when administered to a human, and approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the therapeutic is administered. Such pharmaceutical carriers can be sterile liquids, such as saline solutions in water and oils, including those of petroleum, animal, vegetable, or synthetic origin, such as peanut oil, soybean oil, mineral oil, sesame oil, and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents.

Pharmaceutical compositions adapted for oral administration may be capsules, tablets, powders, granules, solutions, syrups, suspensions (in non-aqueous or aqueous liquids), or emulsions. Tablets or hard gelatin capsules may comprise lactose, starch or derivatives thereof, magnesium stearate, sodium saccharine, cellulose, magnesium carbonate, stearic acid or salts thereof. Soft gelatin capsules may comprise vegetable oils, waxes, fats, semi-solid, or liquid polyols. Solutions and syrups may comprise water, polyols, and sugars. An active agent intended for oral administration may be coated with or admixed with a material that delays disintegration and/or absorption of the active agent in the gastrointestinal tract. Thus, the sustained release may be achieved over many hours and if necessary, the active agent can be protected from degradation within the stomach. Pharmaceutical compositions for oral administration may be formulated to facilitate release of an active agent at a particular gastrointestinal location due to specific pH or enzymatic conditions.

In order to overcome any issue of the pharmacological agents crossing the blood/brain barrier, intrathecal administration is a further preferred form of administration. Intrathecal administration involves injection of the drug into the spinal canal, more specifically the subarachnoid space such that it reaches the cerebrospinal fluid. This method is commonly used for spinal anesthesia, chemotherapy, and pain medication. Intrathecal administration can be performed by lumbar puncture (bolus injection) or by a port-catheter system (bolus or infusion). The catheter is most commonly inserted between the laminae of the lumbar vertebrae and the tip is threaded up the thecal space to the desired level (generally L3-L4). Intrathecal formulations most commonly use water, and saline as excipients but EDTA and lipids have been used as well.

Pharmaceutical compositions adapted for nasal and pulmonary administration may comprise solid carriers such as powders, which can be administered by rapid inhalation through the nose. Compositions for nasal administration may comprise liquid carriers, such as sprays or drops. Alternatively, inhalation directly through into the lungs may be accomplished by inhalation deeply or installation through a mouthpiece. These compositions may comprise aqueous or oil solutions of the active ingredient. Compositions for inhalation may be supplied in specially adapted devices including, but not limited to, pressurized aerosols, nebulizers or insufflators, which can be constructed so as to provide predetermined dosages of the active ingredient.

A further preferred form of administration is parenteral including intravenous administration. Pharmaceutical compositions adapted for parenteral administration, including intravenous administration, include aqueous and non-aqueous sterile injectable solutions or suspensions, which may contain anti-oxidants, buffers, bacteriostats, and solutes that render the compositions substantially isotonic with the blood of the subject. Other components which may be present in such compositions include water, alcohols, polyols, glycerine, and vegetable oils. Compositions adapted for parental administration may be presented in unit-dose or multi-dose containers, such as sealed ampules and vials, and may be stored in a freeze-dried (lyophilized) condition requiring only the addition of a sterile carrier, immediately prior to use. Extemporaneous injection solutions and suspensions may be prepared from sterile powders, granules, and tablets. Suitable vehicles that can be used to provide parenteral dosage forms of the invention are well known to those skilled in the art. Examples include: Water for Injection USP; aqueous vehicles such as Sodium Chloride Injection, Ringer's Injection, Dextrose Injection, Dextrose and Sodium Chloride Injection, and Lactated Ringer's Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate.

Further methods of administration include sublingual, vaginal, buccal, or rectal; or transdermal administration to a subject.

Selection of a therapeutically effective dose will be determined by the skilled artisan considering several factors, which will be known to one of ordinary skill in the art. Such factors include the particular form of the pharmacological agent, and its pharmacokinetic parameters such as bioavailability, metabolism, and half-life, which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether the administration is acute or chronic, concomitant medications, and other factors well known to affect the efficacy of administered pharmaceutical agents. Thus, the precise dose should be decided according to the judgment of the person of skill in the art, and each patient's circumstances, and according to standard clinical techniques.

As shown in the Examples, the effects of one injection of a low dose of topotecan persisted up to four to five months and there was no toxicity seen for nine months.

Doses can be adjusted to optimize the effects in the subject. For example, the topoisomerase type I inhibitor can be administered at a low dose to start and then increased over time to depending upon the subject's response. A subject can be monitored for improvement of their condition prior to changing, i.e., increasing or decreasing, the dosage. A subject can also be monitored for adverse effects prior to changing the dosage, i.e., increasing or decreasing, the dosage.

Topotecan and irinotecan are FDA approved chemotherapeutic agents. Topotecan is marketed in capsules of 1 mg and 0.25 mg in strength and in powder for injection/intravenous administration in 1 mg and 4 mg in strength. Topotecan is generally given in dosages of ranging from 0.75 mg/ml to 1.5 mg/ml including 0.9 mg/ml, 1.0 mg/ml and 1.25 mg/ml daily for five days or 4.0 mg/ml weekly and at these dosages has mild nonhematologic toxicity and reversible manageable hematologic toxicity.

Irinotecan is marketed in vials containing 40 mg and 100 mg and is generally given in dosages of 125 mg/ml and 350 mg/ml. It is only administered intravenously.

The agents described herein can be co-administered with other agents. The co-administration of agents can be by any administration described herein. Moreover, it can be in one composition, or in more than one composition. The administration of the agents can be simultaneous, concurrently or sequentially.

### Kits

Also within the scope of the present disclosure are kits for practicing the method of the invention. Such kits may include agents that activates or increases the expression and/or activity of Ube3A for the prevention and/or treatment of diseases characterized by synaptic dysfunction and neurodegeneration including but not limited to Alzheimer's disease.

In some embodiments, the kit can comprise instructions for use in any of the methods described herein. The included instructions can comprise a description of administration of the agents to a subject to achieve the intended activity in a subject. The kit may further comprise a description of selecting a subject suitable for treatment based on identifying whether the subject is in need of the treatment.

The instructions relating to the use of the agents described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### Drug Screening and Research Tools

A further embodiment of the invention is the use of Ube3A and the methods set forth herein as a method for screening for potential therapies for diseases characterized by synaptic dysfunction and neurodegeneration, especially Alzheimer's disease.

In one embodiment, the DNA or mRNA encoding the Ube3A protein is used. In one embodiment, the nucleic acid can be cloned into any of the vectors described herein by the methods described herein. The recombinant constructs can also have a means to measure expression of expression, such as a reporter construct. The recombinant constructs comprising the DNA or mRNA encoding the Ube3A protein are then contacted with the agent, and if the expression of the gene linked to Ube3A increases, then the agent can be considered a potential therapy.

These recombinant constructs can be transfected into any host cell described herein, by the methods described herein.

The host cells with the recombinant constructs can then be used for screening for therapeutic agents for diseases characterized by synaptic dysfunction. In this embodiment, the expression of Ube3A is measured by the expression of the linked gene prior to the contact with the agent. The transformed host cells are then contacted with the agent, and if the expression of the gene linked to Ube3A increases, then the agent can be considered a potential therapy.

Any of the constructs and transformed host cells can also be used for basic research related to AD, and other diseases characterized by synaptic dysfunction and/or neurodegeneration.

These gene constructs as well as the host cells transformed with these gene constructs can also be the basis for transgenic animals for testing both as research tools and for therapeutic agents.

### EXAMPLES

The present invention may be better understood by reference to the following non-limiting examples, which are presented in order to more fully illustrate the preferred embodiments of the invention. They should in no way be construed to limit the broad scope of the invention.

### Example 1—Materials and Methods for Examples 2-12

Materials.

All chemicals used were of the highest grade available. Antibodies used include the following: rabbit anti-Ube3A (Cell Signaling), mouse anti-RhoA (Santa Cruz), rabbit anti-Arc (Santa Cruz), mouse anti-GluR1 (Millipore), rabbit anti-Ephexin-5 (Pierce and Abcam), rabbit anti-phospho-c-Abl (Cell Signaling), rabbit anti-GAPDH (Cell Signaling), rabbit anti-HA (Cell Signaling), anti-PSD95 (Cell Signaling), rabbit anti-NMDAR1 (Millipore), and anti-phospho-tau PHF13 (Cell Signaling).

Primary Hippocampal Neuronal Cultures.

Hippocampal neuron cultures from both male and female rat embryos (E17-18) were prepared following a slightly modified version of the method of Brewer et al. 1996. Hippocampal neurons were kept in culture at 37° C. with 5% CO2 in Neurobasal medium (ThermoFisher) with B27 (ThermoFisher) supplement and Glutamax (ThermoFisher) and plated at a density of 2.5×10⁵ cell/ml on dishes coated with poly-D-lysine (ThermoFisher). For the experiments, neurons were used after 14-21 days in vitro (DIV). Rat neuroblastoma B35 cells were cultured and maintained in DMEM (ThermoFisher) supplemented with 10% fetal bovine serum (FBS; Invitrogen) and Glutamax (ThermoFisher).

Penetratin-1-Mediated Delivery of siRNA.

Oligonucleotide sequences were custom-ordered (Dharmacon) with a thiol functionality at the 5′-end (Ephexin-5 siRNA sequences—Sense: GUCCUUUCUCCGUGUAUGUUU (SEQ ID NO: 1); Antisense: ACAUACACGGAGAAAGGACUU (SEQ ID NO: 2)).

Oligonucleotide stocks were solubilized in DNase/RNase-free water (ThermoFisher) at a 10 mM concentration. The delivery peptide Penetratin-1 (MP Biomedicals) was cross-linked via a Cys-Cys bond to the desired oligonucleotide as previously described (Davidson et al. 2004).

Transgenic Mice.

The Tg2576 mouse model (Hsiao et al. 1996) used in this study was originally generated by Karen Hsiao and overexpresses a mutant form of APP (isoform 695) with the Swedish mutation (KM670/671NL).

The J20 transgenic mouse line also used in the studies expresses a mutated human APP (hAPP: K670N/M671L and V717F) under the control of the platelet-derived growth factor promoter (Mucke et al. 2000). These mice have deficits in learning and memory, inhibition of long-term potentiation, loss of dendritic spines, profuse amyloid plaques, and increased phosphorylation of the tau protein. These mice also have increased RhoA-GTP activity and progressive decrease in spinal density when treated with oligomeric Aβ. See Pozueta et al. 2013.

Male mice were kept under standard housing conditions (12 h light/dark cycle) with ad libitum access to food and water. Age-matched wild-type (WT) littermates were used as controls. Hippocampi were dissected after euthanasia at 9-10 months of age.

All animal usage was performed under the guidelines of the Columbia University Institutional Animal Care and Use Committee (IACUC).

Morris Water Maze.

Mice were trained to find a hidden platform in a circular pool of 1.10 m diameter located in a room with extra maze cues. The location of the platform (14 mm diameter) was constant for each mouse during training and was placed 1 cm beneath the surface of the water, maintained at 24° C.±2° C. throughout the duration of the test. At the end of test, the mice were dried off and placed in a clean cage with extra paper towels to prevent hypothermia Animals were then monitored inside their cage until eating, drinking, and ambulating normally Mice were trained in sessions consisting of four trials a day for five consecutive days. Mice started from different quadrants on a random basis during each trial and all four quadrants were used on any given day. The maximum swimming time allowed on each trial was 60 seconds. If the mouse failed to reach the platform within 60 seconds, it was manually guided to the platform and kept there for 15 seconds.

Probe trials (1 session: 1 trials/session) were conducted 24 hours after the last training trial. During the probe test the platform was removed and mice were free to swim in the pool for 60 seconds. Time spent in each quadrant was measured. Trial and probe test sessions were recorded and analyzed with a video tracking system (Noldus). After the probe, mice were tested in the visible test for two consecutive days, 2 sessions/day with 3 trials/session to assess whether the mice were able to locate a visible platform (its location varied between the three trials of the same session). The ability of the mice to locate a clearly visible platform was also tested in order to exclude differences in vision, swim speed, and motivation.

Oligomeric oAβ Preparation.

Lyophilized oAβ (rPeptide) was dissolved to 1 mM in hexafluoro-2-propanol (HFIP; Sigma) for 1 hour in a sterile hood. The dissolved peptide solution was then aliquoted into Teflon-coated tubes and HFIP was allowed to evaporate at room-temperature overnight under sterile conditions. The resulting pellet was further dried in a SpeedVac for 1 hour at room temperature. Tubes were sealed and stored then at −30° C. freezer with desiccator pellets. To prepare oligomeric oAβ, the pellet was resuspended in DMSO at 5 mM and sonicated for 15 minutes at room temperature. The solution was further diluted in DMEM/F12 to 100 μM and allowed to oligomerize at 4° C. for 24 hours. For simplification, oligomeric oAβ will be referred to in the text as oAβ.

DiOlistic Labeling of Neurons for Dendritic Spine Analysis.

The protocol for diolistic labeling of neurons was adapted from Gan et al. 2000 and Gan et al. 2009. Briefly, 100 mg of tungsten particles (1.1 μm diameter; Bio-Rad) were thoroughly precipitated with 5.0 mg of lipophilic dye (DiI; ThermoFisher) and dissolved in 100 μl of methylene chloride. Dye-coated particles were shot 2-3 times into the cells using the Helios gene gun system (Bio-Rad) and left in 0.1 M PBS overnight to allow dye diffusion along neuronal processes. Cells were post-fixed with 4% paraformaldehyde (Pierce) for 1 hour to preserve staining, then mounted onto glass slides using Gel Mount (Biomeda) and stored at 4° C. in the dark.

Spine Imaging, Quantification, and Statistics.

Labeled neurons were imaged using the Laser Scanning Microscope (LSM) 510 Meta confocal microscope (Zeiss), equipped with 40×1.3 NA and 100×1.4 NA oil-immersion objectives. The NIH Image software program ImageJ was used the quantify diolistic-labeled cultured neurons. An average of 12 compressed images (20 μm thick) consisting of pyramidal neurons were quantified for each treatment. Spines were counted with NeuronStudio software in a blinded manner and spine density was represented in number of spine per μm dendritic length. Statistical analysis was performed using the statistics package GraphPad Prism.

Immunofluorescence and Microscopy.

Cells were fixed in PFA (4%) in 590 PBS for 10 minutes at room temperature. Permeabilization was performed using 0.4% Triton X-100 (Sigma) in PBS for 20 minutes. Then, cells were incubated in anti-GluR1 primary antibody (Millipore) in Superblock (Thermo Scientific) with 0.1% Triton X-100 for 12 hours at 4° C. Cells were washed with PBS, and then incubated in goat anti-mouse Alexa 568 in Superblock with 0.1% Triton X-100 for 1 hour at RT. Then cells were mounted onto glass slides using Gel Mount (Biomeda).

Immunoprecipitation (IP).

Cells were collected and homogenized with IP lysis buffer (ThermoFisher) supplemented with Halt protease and phosphatase inhibitor (ThermoFisher). Cell extracts were incubated overnight at 4° C. with 1.0 μg HA antibody (Cell Signaling) or mouse IgG (Cell Signaling) as a negative control Immunocomplexes were incubated with magnetic beads covalently coated with protein G (Invitrogen) on a rotator for 2 hours, at 4° C. The beads were then pelleted with a magnet and washed 3 times with 1× cell lysis buffer (Cell Signaling) supplemented with complete mini protease inhibitor mixture (Roche). The immunocomplexes were resuspended in cell lysis buffer, LDS-sample buffer (Invitrogen), 50 mm dithiothreitol (DTT), and analyzed by Western blotting.

Sample Preparation for Immunoblotting Analysis.

Whole frozen mouse hippocampi, human brain specimens, and cultured cells were homogenized in RIPA buffer (ThermoFisher) supplemented with protease and phosphatase inhibitor cocktail (ThermoFisher), using a Teflon dounce homogenizer and centrifuged at 14,000 rpm for 10 minutes at 4° C. Supernatants were collected, normalized by BCA assay (ThermoFisher), and prepared for loading using sample buffer (SB) 4× (ThermoFisher) and dithiothreitol (DTT) 10× (ThermoFisher).

qPCR.

Neurons were lysed and total RNA was extracted using TRIzol reagent (Ambion) following the manufacturer's protocol. RNA concentration and purity were assessed by measuring the optical density at 260 and 280 nm with a NanoDrop (Thermo Scientific). cDNA was synthesized using the first-strand cDNA synthesis kit (Origene) with 1 μg of total RNA, following the manufacturer's instructions. Quantitative real-time PCR was performed using FastStart SYBR Green Master Mix (Roche) and an Eppendorf Realplex Mastercyler with the following cycle settings: 1 cycle at 95° C. for 10 min and 40 cycles of amplification, 95° C. for 15 s, 58-60° C. for 30-60 s, 72° C. for 30-60 s. The amounts of Ube3A mRNA were quantified and normalized to GAPDH mRNA using the following primer pairs: Ube3A forward 5′-CGAGGACAGATCACCAGGAG-3′ (SEQ ID NO: 3) and reverse 5′-TCATTCGTGCAGGCCTCATT—3′ (SEQ ID NO: 4). The threshold cycles were determined and normalized to the threshold cycles of a GAPDH gene. Relative mRNAs levels for Ube3A were expressed as a fold induction in an experimental condition compared with a control condition.

Rho-GTPase Activity Assay.

Rho-GTPase pull-down assays were performed following the manufacturer's protocol (Cytoskeleton, Inc.). Briefly, cells were washed once with ice-cold PBS and lysed with RIPA/M-PER buffer (1:1; ThermoFisher). Lysates were incubated on ice for 5 minutes, and centrifuged (12,000 g) for 5 minutes. 30 μl was used for input and the remaining lysates were incubated with Rhotekin-RBD (RhoA) or PAK-RBD (Rac1) protein beads (25 μl) by rotating for 1 hour at 4° C. Beads were washed with ice-cold PBS (500 μl, 3×) and collected, and bound proteins were eluted with 1× sample buffer (30 μl). Input and eluted samples were then analyzed by gel electrophoresis and Western blotting using either a RhoA or Rac1-specific antibody.

Electrophysiological Analyses.

Long-term potentiation (LTP) recordings were performed by positioning glass electrodes filled with artificial cerebrospinal fluid (ACSF) in the CA1 stratum radiatum to record Field Excitatory Post-Synaptic Potentials (fEPSPs) evoked by local stimulation (0.1 ms) of Schaffer collateral fibers using a bipolar concentric electrode placed laterally to the recoding electrode (approximately 150 μm). Following a steady baseline of 15 minutes, potentiation was induced with three theta-burst stimulations (TBS; 15 second interval), each one involving a single train of 10 bursts at 5 Hz, where each burst was composed of four pulses at 100 Hz (strong stimulation). The fEPSP slopes following tetanic stimulation were normalized to the average of the slopes of the fEPSPs acquired during the baseline.

Histological Studies.

Immunolabeling was done on 30 μm brain sections from the J20 TOP treated mice and PBS treated wild-type and J20 mice. Staining was done using the anti-Aβ antibody 6E10 (Covance) and GFAP (Cell Signaling). Plaque load and astrocytosis were quantified using ImageJ. Total plaque area was expressed as a percentage of the total hippocampal area.

Statistics.

All statistical analyses were done using GraphPad Prism software. Statistical significance was determined by student t-test or one-way analysis of variance (ANOVA), where appropriate, with a significant threshold of p<0.05. Data are expressed as mean±SEM or SD, as appropriate.

### Example 2—Ube3A Expression was Decreased in Tg2576 Hippocampus and Correlated with Upregulation of its Substrates, Ephexin-5 and Arc

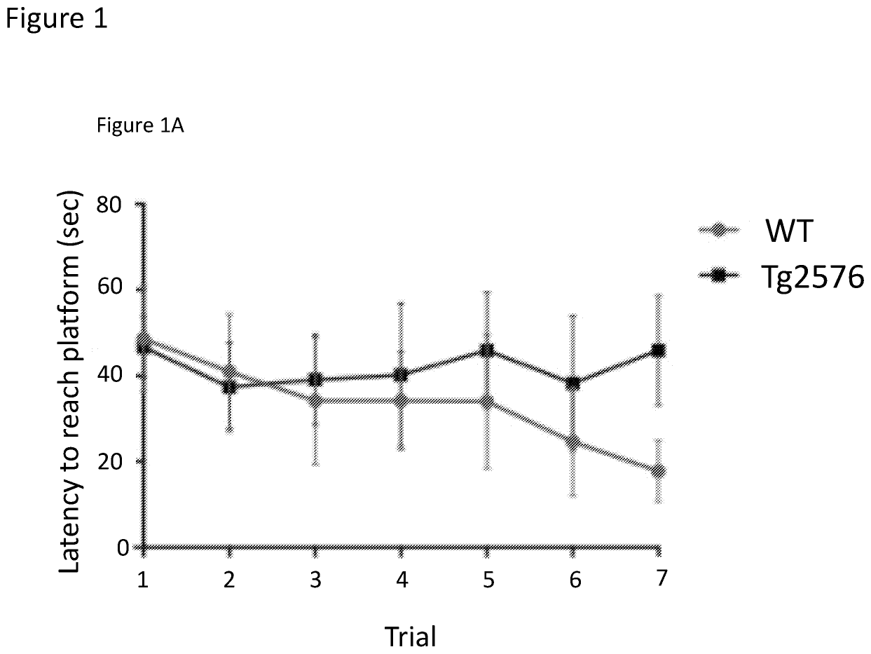

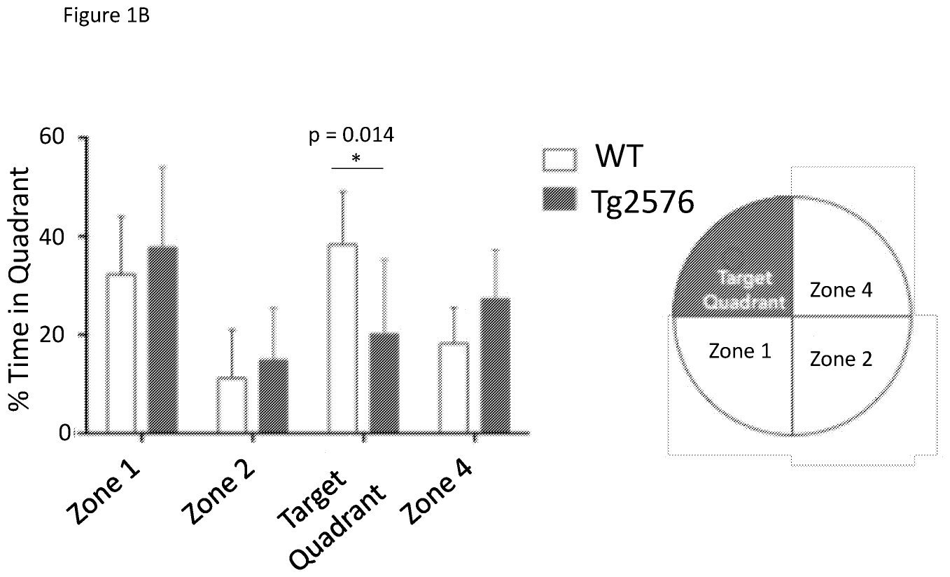

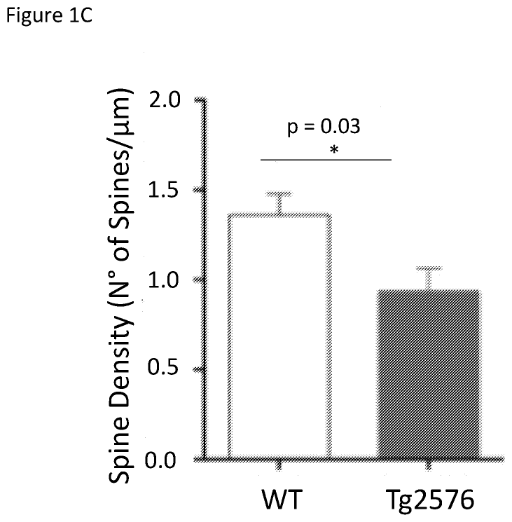

The Tg2576 AD mouse model (Hsiao et al. 1996) overexpresses hAPP carrying the Swedish mutation (KM670/671NL), linked to early-onset familial AD (EOFAD). As previously shown, the Tg2576 mice developed amyloid plaques and age-dependent progressive behavioral deficits as measured by their inability to learn and to recall the location of a hidden platform in the Morris water maze (MWM) task, evident by 12-14 months of age, compared to wild-type (WT) littermates (FIGS. 1A and 1B). No differences in swimming speeds and ability to locate a visible platform were observed between WT and Tg2576 mice (results not shown). The behavioral deficits in Tg2576 mice were accompanied by a significant decrease in dendritic spine density in the hippocampus, observed by 9-10 months of age (FIG. 1C).

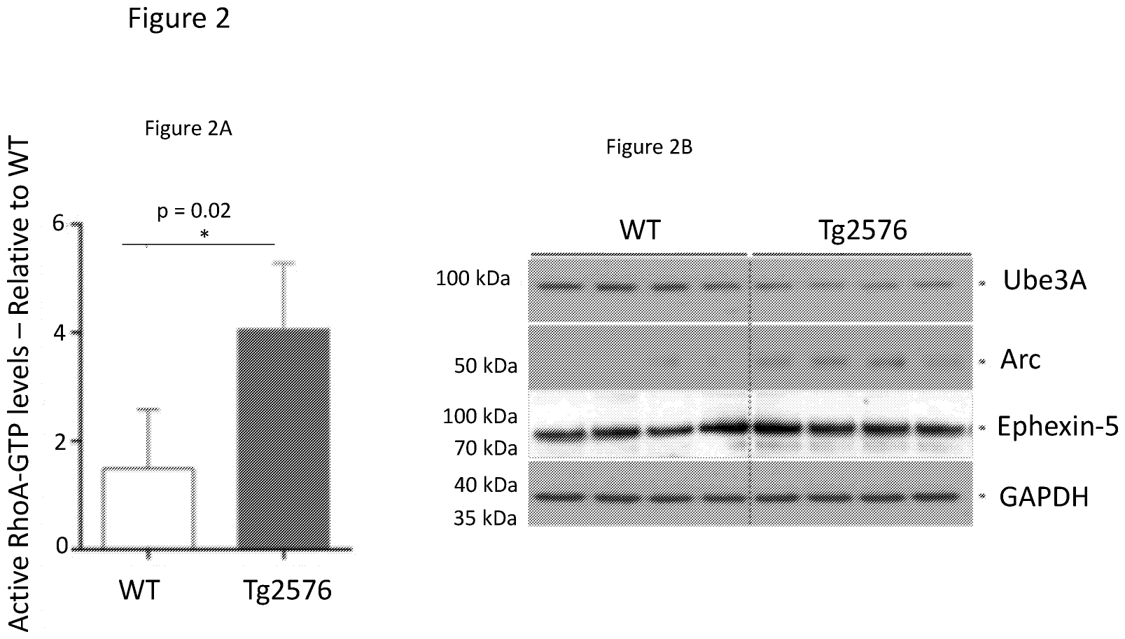

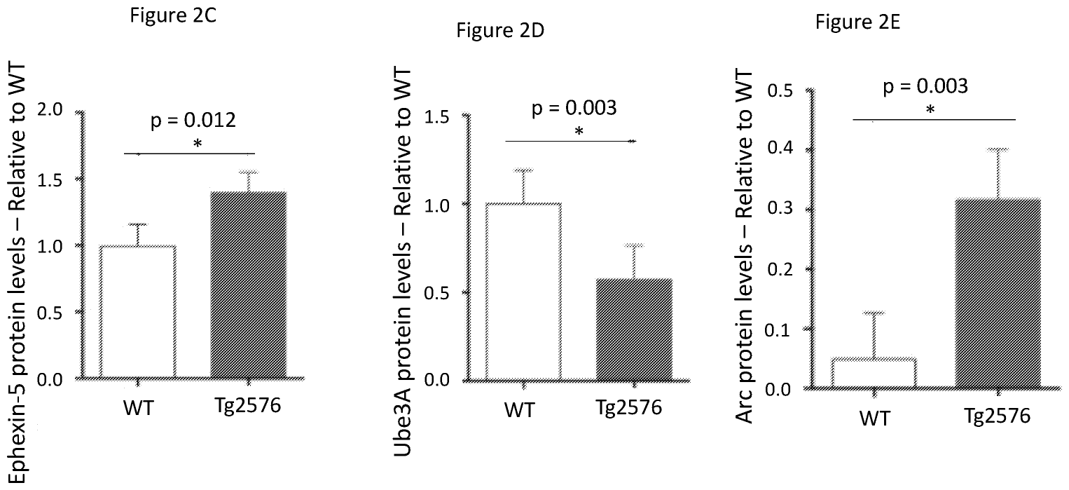

As previously shown, there is a loss of dendritic spine density in the J20 mouse model accompanied by an increase in RhoA activity (Pozueta et al. 2013). To determine whether this was also the case in the Tg2576 mice, whole hippocampi were collected at 9-10 months of age and RhoA activity was assessed. A significant increase in the levels of active RhoA (RhoA-GTP) in Tg2576 was found as compared to WT mice (FIG. 2A).

The activity of RhoA, like other GTPases, is regulated by GEFs, which catalyze the exchange of bound guanosine-5′-diphosphate (GDP) for guanosine-5′-triphosphate (GTP), and inactivated by GTPase activating proteins (GAPs), which catalyze the hydrolysis of GTP to GDP (Pertz 2010). One of the critical neuronal RhoA-specific GEFs is Ephexin-5, which plays an important role in neuronal development and maturation by negatively regulating synapse formation (Margolis et al. 2010). Interestingly, recent evidence suggest that Ephexin-5 may be upregulated in AD brains and in AD mouse models (Sell et al. 2017). Similarly, a significant increase in the levels of Ephexin-5 were found in Tg2576 hippocampi at 9-10 months of age (FIGS. 2B and 2C).

The levels of Ephexin-5 are regulated through proteasomal degradation. Activation of the erythropoietin-producing hepatocellular (Eph) family of receptor tyrosine kinases, EphB2, results in ubiquitination of Ephexin-5, catalyzed by Ube3A (Margolis et al. 2010). While there was no difference observed in the levels of EphB2 protein by Western blot in the hippocampi of Tg2576 mice at 9-10 months of age, a significant decrease in the levels of Ube3A protein was found (FIGS. 2B and 2D).

The levels of Arc, another potential important Ube3A substrate in neurons, were also analyzed and it showed a significant decrease in Tg2576 hippocampi compared to WT mice (FIGS. 2B and 2E).

Together, these results showed an inverse correlation between the levels of Ube3A and those of two of its downstream targets, namely Ephexin-5 and Arc, which are hypothesized to account for the loss of dendritic spine density and synaptic dysfunction observed in the Tg2576 mice.

### Example 3—Ube3A Level was Decreased in Hippocampal Neurons in Response to Exogenous Aβ Oligomers (oAβ)

An in vitro model of Aβ1-42 synaptotoxicity was used to further establish a causal relationship between altered levels of Ube3A and those of Arc and Ephexin-5.

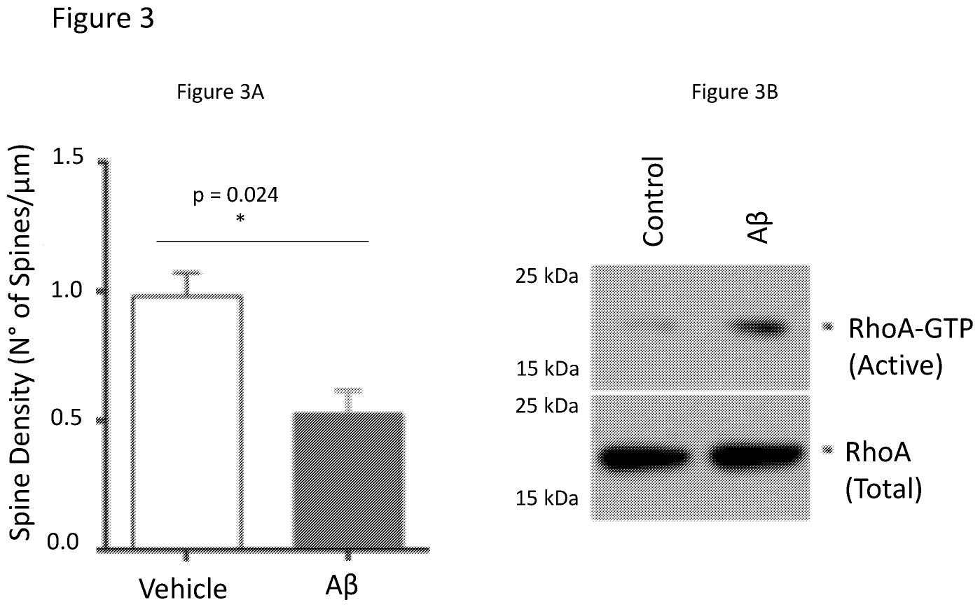

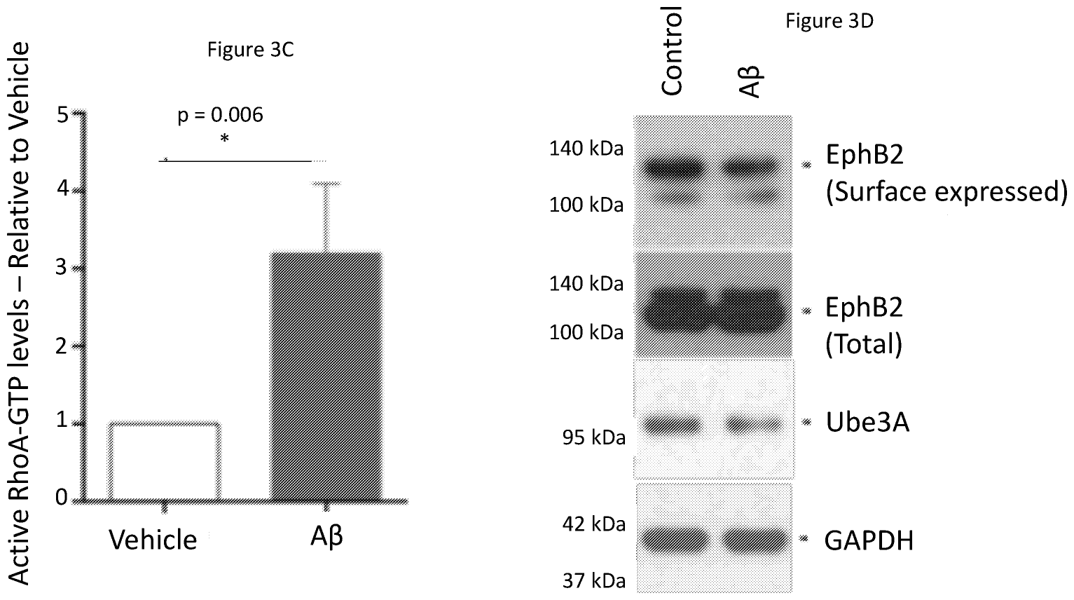

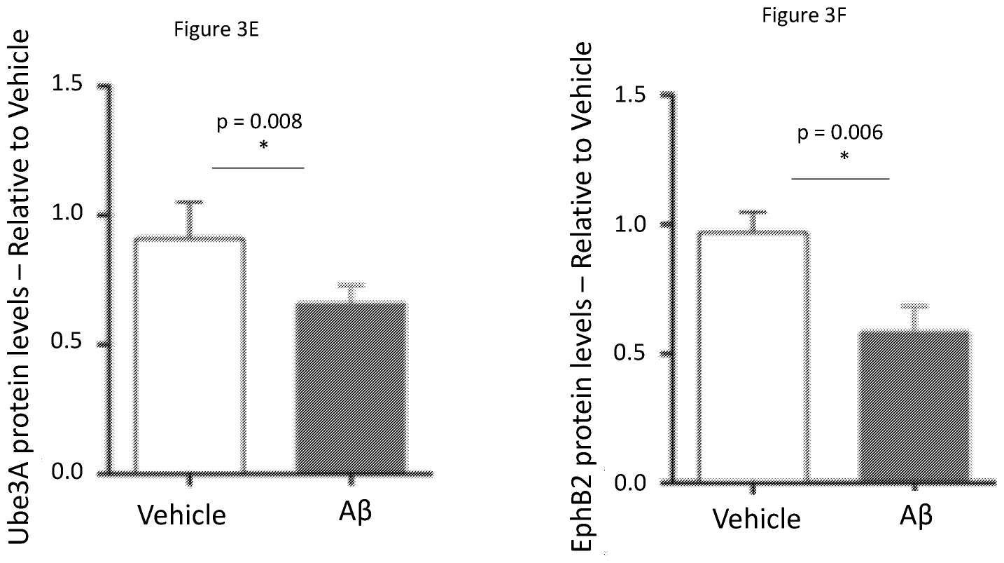

Enriched primary rat hippocampal neurons, cultured for 21 days in vitro, were treated with synthetic soluble oligomeric Aβ1-42 (hereinafter “oAβ”) for 24 hours. No cell death was detected under these conditions (data not shown). However, consistent with the findings in the Tg2576 mice, a significant decrease in dendritic spine density was observed (FIG. 3A), concomitant with a significant increase in the levels of active RhoA-GTP (FIGS. 3B and 3C). Moreover, also consistent with the findings in the Tg2576 mice, Western blot analysis revealed a significant decrease in the levels of Ube3A protein (FIGS. 3D and 3E).

Interestingly, as previously shown (Cisse et al. 2011), a decrease in the levels of surface expressed EphB2 protein was detected (FIGS. 3D and 3F), suggesting that the loss of EphB2 may also facilitate the accumulation of Ephexin-5. Since Ube3A is also expressed in astrocytes (Grier et al. 2015), it was determined whether Ube3A levels were also altered in these cells. Contrary to primary hippocampal neurons, treatment of astrocyte-enriched cultures with oAβ resulted in a modest, but significant increase in Ube3A levels, suggesting that the oAβ-induced decrease in Ube3A is specific to neurons (results not shown).

### Example 4—oAβ Induced c-Abl-Dependent Tyrosine-Phosphorylation and Destabilization of Ube3A Protein in Hippocampal Neurons

In mammals, the Ube3A gene is subject to genomic imprinting, whereby only the maternal allele is expressed in neurons. The paternal allele is silenced through the expression of a Ube3A-antisense (Ube3A-ATS), transcribed as part of a large non-coding antisense transcript (Runte et al. 2001). It was first determined whether oAβ affected the levels of Ube3A mRNA. qPCR analysis of oAβ-treated hippocampal neurons for 24 hours did not show any differences in the levels of Ube3A mRNA (results not shown).

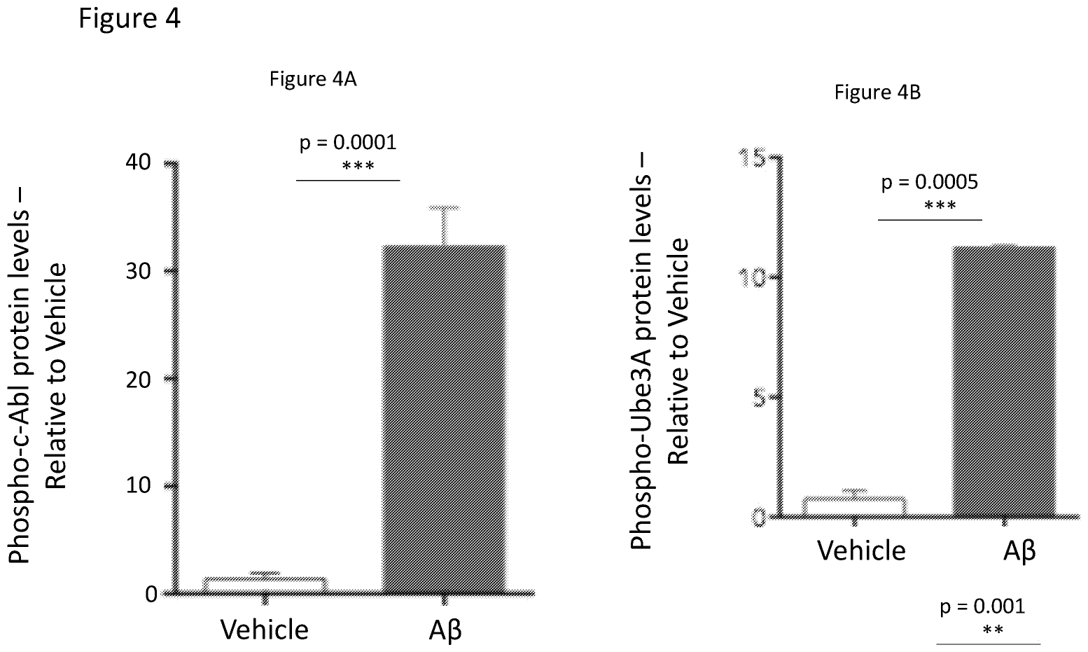

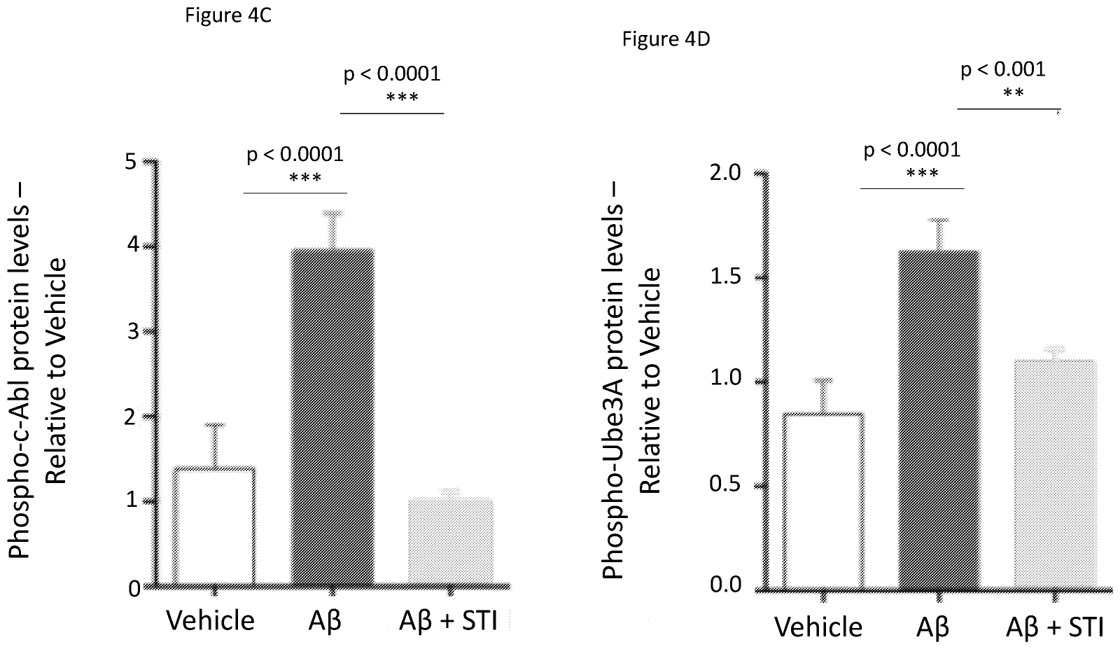

It was hypothesized that oAβ could affect the ligase function of Ube3A. One important regulatory mechanism of Ube3A is through c-Abl-mediated phosphorylation tyrosine residue 636 (Y636), which impairs its E3 ligase activity (Chan et al. 2013). Interestingly, c-Abl has previously been implicated in AD pathogenesis and has been shown to be activated in AD brains and in AD mouse models (Vargas et al. 2014), including in the Tg2576 mice. Since phospho-Ube3A antibodies are not available it was decided to test the hypothesis in B35 neuroblastoma cell line overexpressing HA-tagged Ube3A for immunoprecipitation. Consistent with the hypothesis, it was found that treatment of these cells with, either the c-Abl activator, DPH, used as a positive control (FIGS. 4A and 4B), or with oAβ (FIGS. 4C and 4D), resulted in a significant increase in phospho-tyrosinated Ube3A (pTyr-Ube3A), as early as 1 hour, as determined by immunoblotting analysis of immunoprecipitated HA-Ube3A with a phospho-tyrosine-specific antibody. To confirm the involvement of c-Abl in tyrosine-phosphorylation of Ube3A, cells were pre-treated with the specific c-Abl inhibitor, STI-571 (Schindler et al. 2000; Buchdinger et al. 1996) for 1 hour prior to oAβ treatment. Inhibition of c-Abl resulted in the complete abolishment of Ube3A phosphorylation by oAβ (FIGS. 4C and 4D).

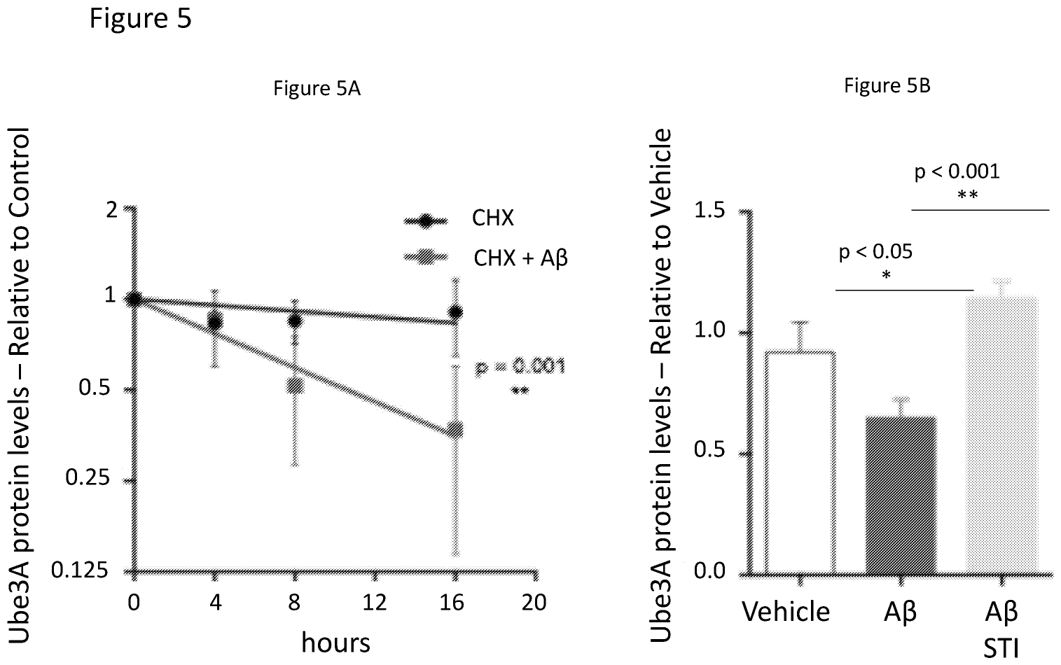

Interestingly, these results suggested that tyrosine-phosphorylation of Ube3A results in destabilization of the protein. Under baseline conditions, Ube3A protein had a half-life of greater than 24 hours, as determined by cycloheximide (CHX) chase assay (FIG. 5A). However, treatment with oAβ significantly increased the degradation rate of the Ube3A protein (FIG. 5A). The decrease in Ube3A protein was completely blocked by pre-treatment with STI, consistent with the role of c-Abl in mediating Ube3A inactivation and degradation (FIG. 5B). These results were consistent with previous studies which showed that STI treatment completely blocked the synaptotoxic effects and the loss of dendritic spines induced by oAβ (Vargas et al. 2014).

### Example 5—oAβ-Induced Dysfunction of Ube3A Correlated with Increased Levels of Arc and Ephexin-7 in Hippocampal Neurons

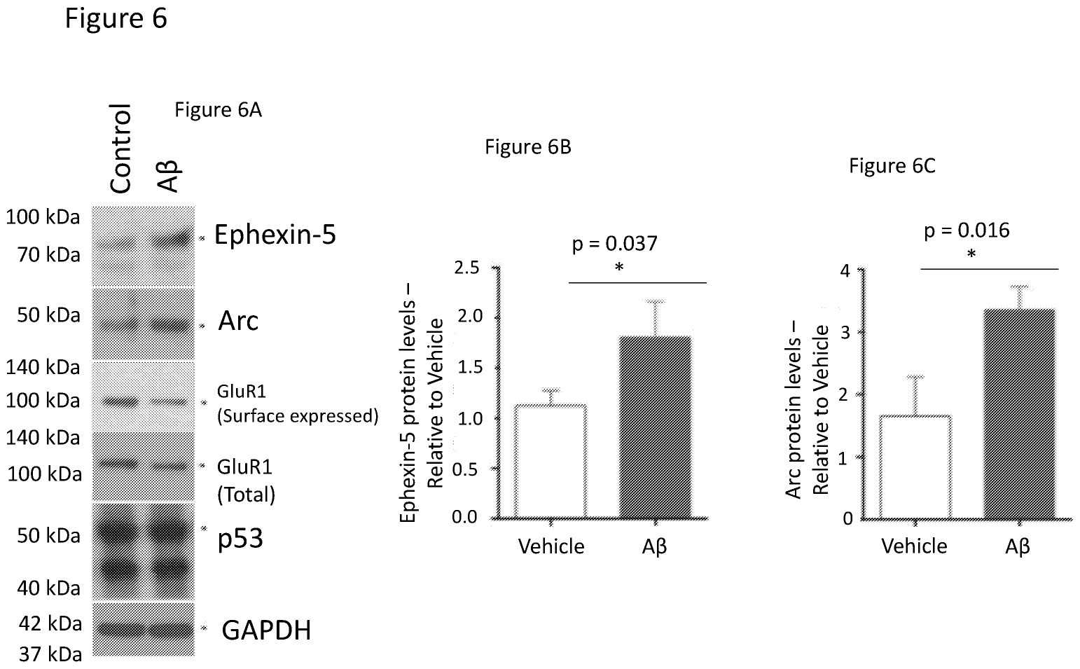

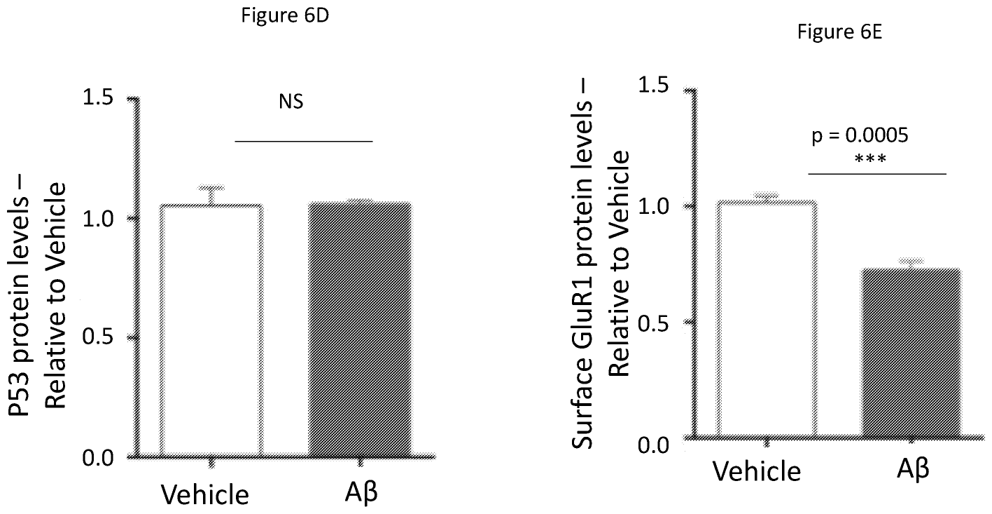

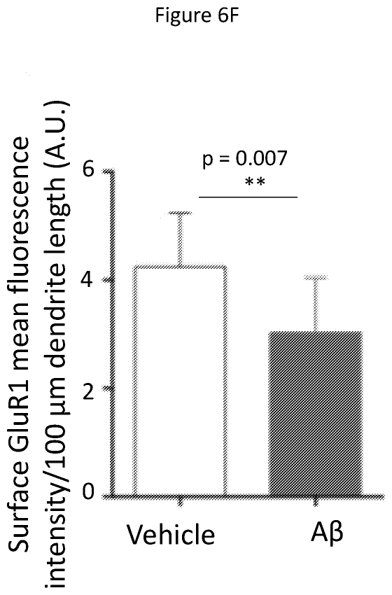

As observed in the Tg2576 mice hippocampi, the levels of Ube3A inversely correlated with the levels of Arc and Ephexin-5. To determine whether this correlation was also observed in hippocampal neurons, the cells were treated with Aβ for 8-10 hours, and whole cell lysates were collected for Western blot analysis. This revealed a significant increase in the levels of both Ephexin-5 and Arc protein (FIGS. 6A, 6B, and 6C). Intriguingly, analysis of another candidate Ube3A-subtrate, p53, did not show any altered levels after Aβ treatment (FIGS. 6A and 6D).

The consequences of upregulation of Arc and Ephexin-5 are well characterized. In the case of Arc, one its key function is to regulate postsynaptic trafficking of AMPA receptor subunits, and to regulate spine morphology and network stability (Korb and Finkbeiner 2011). Aberrant upregulation of Arc has been shown to induce a decrease in surface expression of the AMPA receptor subunit GluR1 (Chowdhury et al. 2006). As previous studies have shown, when hippocampal neurons were treated with Aβ, inactivation of Ube3A correlated with a significant decrease in the levels of surface-expressed GluR1 subunit, as determined by Western blot analysis of isolated biotinylated surface GluR1 (FIGS. 6A and 6E), and by immunostaining of surface GluR1 (FIG. 6F).

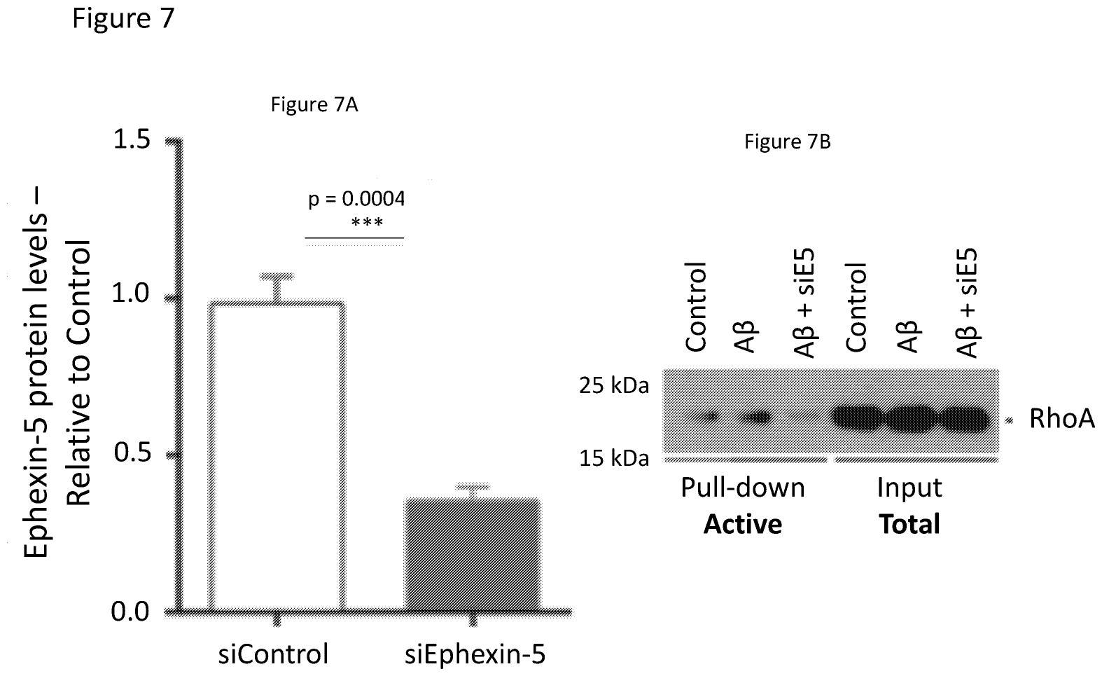

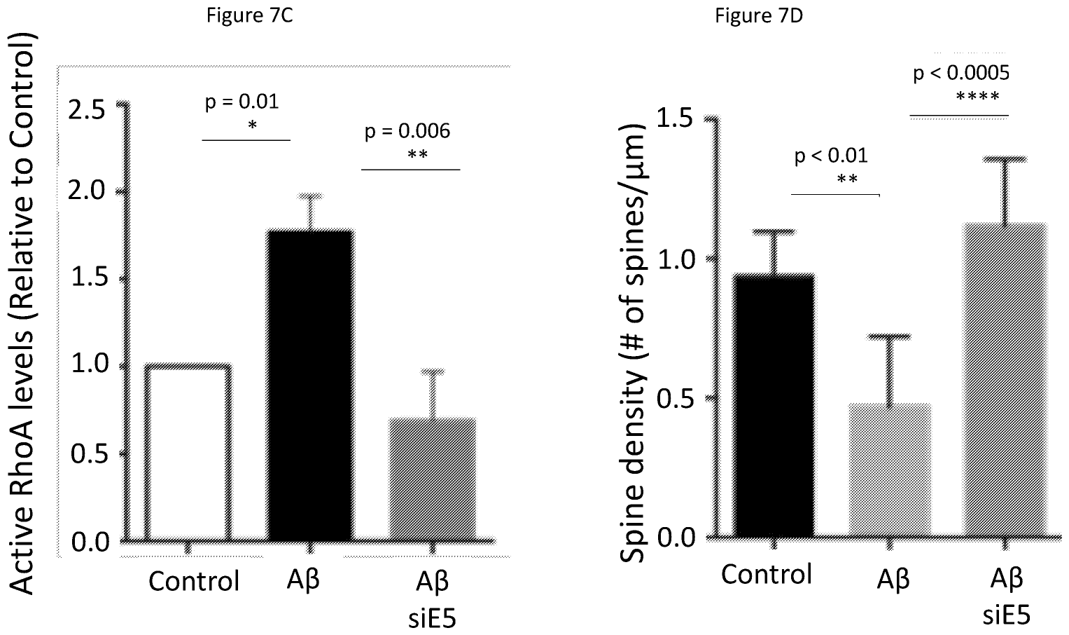

Ephexin-5 on the other hand is a key RhoA-specific GEF in neurons, whose activity is critical for spine formation during development (Margolis et al. 2010). Upregulation of Ephexin-5 leads to aberrant RhoA activation and loss of dendritic spine density. oAβ leads to increased RhoA activation in neurons (Example 2). To confirm that Ephexin-5 is responsible for upregulation of RhoA activity in this model, the levels of Ephexin-5 were depleted by Penetratin-1-linked siRNA in cultured hippocampal neurons for 24 hours (FIG. 7A), prior to treating them with oAβ for 8 hours. Depletion of Ephexin-5 completely blocked the oAβ-induced upregulation of RhoA activity (FIGS. 7B and 7C). Moreover, downregulation of Ephexin-5 completely rescued the loss of dendritic spine normally observed in hippocampal neurons treated with oAβ after 24 hours (FIG. 7D). These data suggested that increased RhoA activity was dependent on the upregulation of Ephexin-5. Consistent with this, there was no observation of any differences in the levels, or activities of two other neuronal RhoA-specific GEFs, namely Ephexin-1 (Shamah et al. 2001) and Lfc (Ren et al. 1998) (results not shown).

### Example 6—Restoring Ube3A Levels Using Gene Delivery Protected Against oAβ-Induced Synaptotoxicity

To further confirm that the loss of Ube3A function plays a key role in mediating oAβ-induced synaptic dysfunction in neurons, the levels of Ube3A protein were restored, through lentiviral mediated upregulation of a Ube3A gene. To carefully regulate transgene expression in neurons, the Ube3A construct was fused with a destabilizing domain (DD), derived from the E. coli dihydrofolate reductase which results in fast proteasomal degradation of the entire fusion protein in the absence of a stabilizing agent (Tai et al. 2012; Iwamoto et al. 2010), in this case the small molecule trimethoprim (TMP). Once TMP is added, it binds to DD and stabilizes the protein, allowing it to accumulate in the cell. In this system, infection of hippocampal neurons with a DD Ube3A-expressing lentivirus resulted in undetectable expression of exogenous Ube3A in the absence of TMP. However, the addition of TMP to the culture medium resulted in a dose dependent increase in DD-Ube3A after 24 hours.

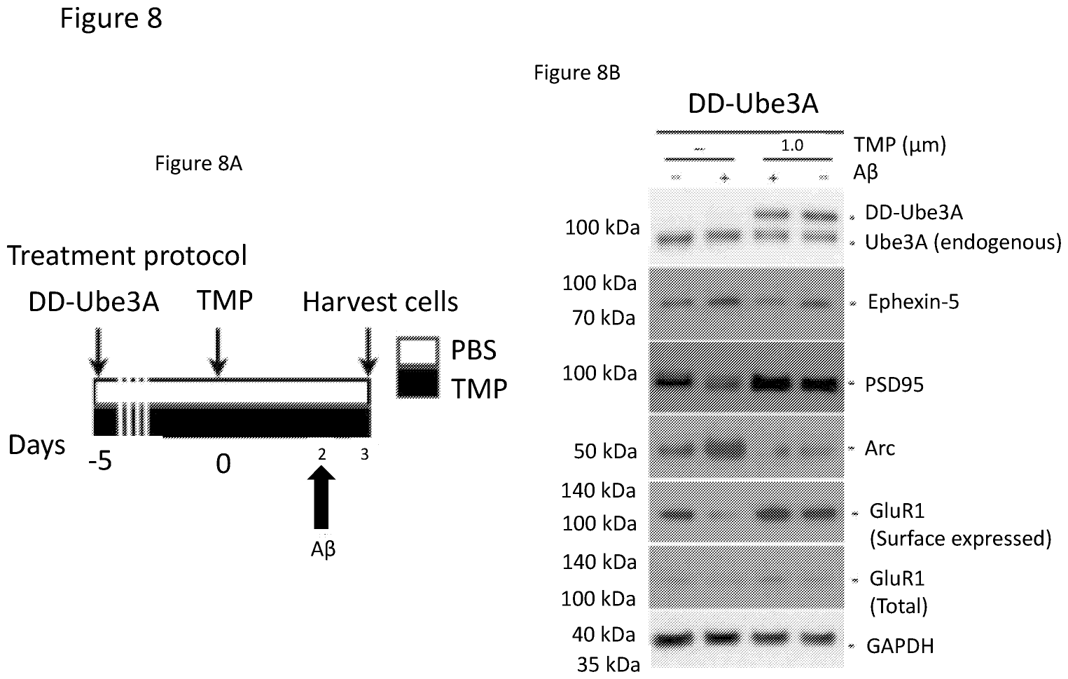

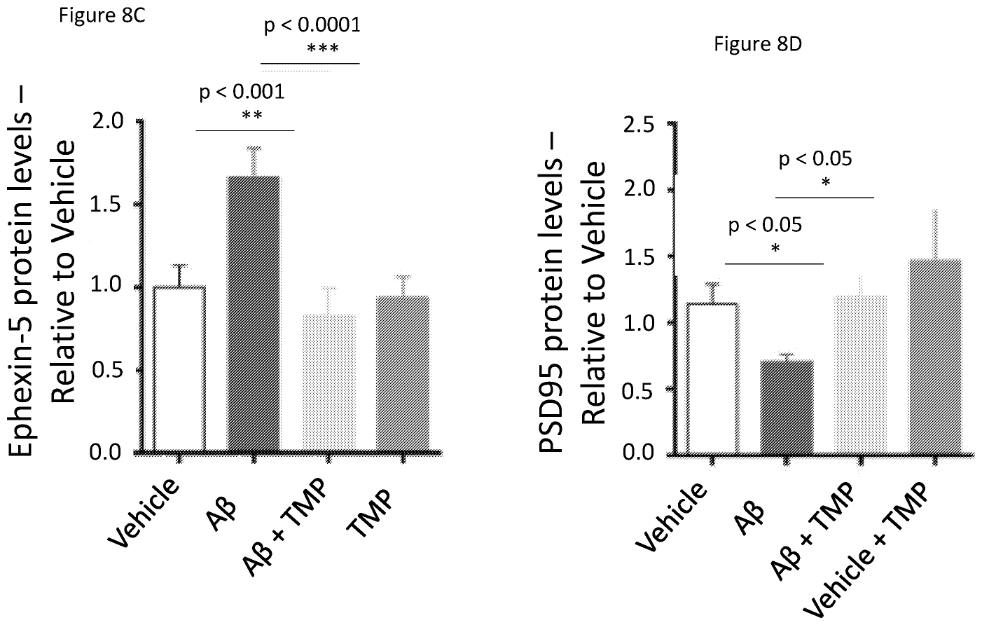

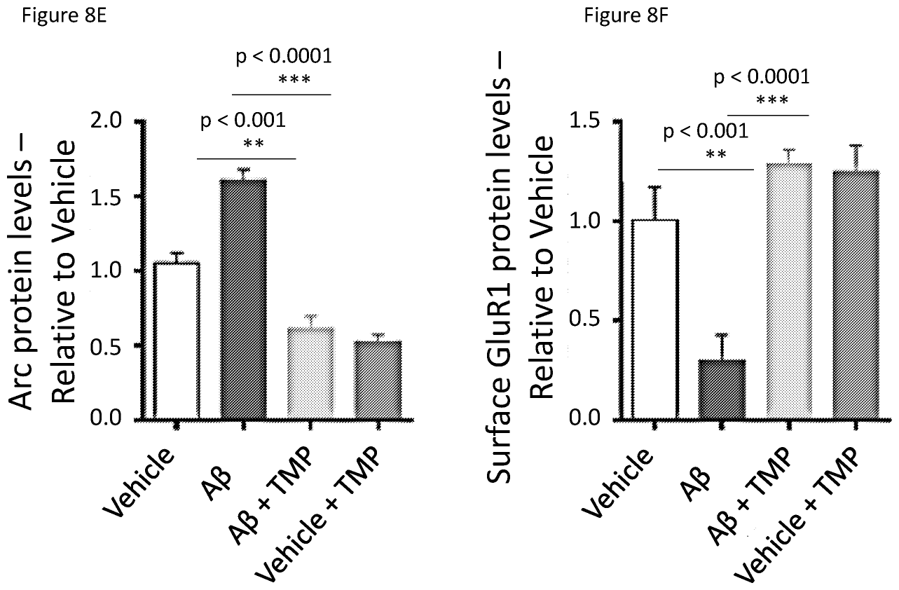

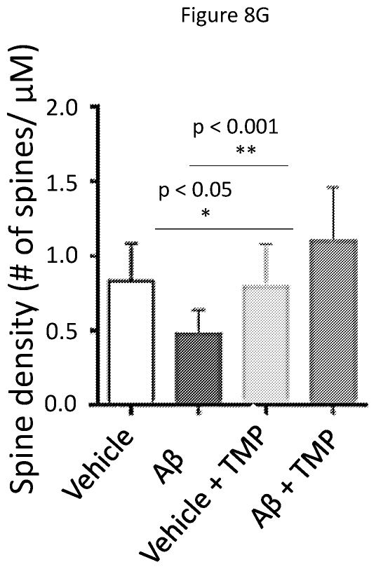

To test whether upregulation of Ube3A could rescue the effects of oAβ, cultured hippocampal neurons at 14 DIV were infected with DD-Ube3A-expressing lentivirus for 5 days, followed by TMP (trimethoprim) treatment for 24 hours or Vehicle. The neurons were then treated with oAβ for 24 hours, and whole cell lysates prepared for Western blot analysis, or fixed for microscopy (FIG. 8A).

Upregulation of Ube3A in oAβ-treated neurons completely blocked the upregulation of Arc and Ephexin-5 (FIGS. 8B, 8C and 8E). Preventing upregulation of Arc also completely blocked the loss of surface GluR1 by oAβ (FIGS. 8B and 8F). Additionally, dendritic spine analysis in fixed neurons revealed that prevention of Ephexin-5 induction resulted in a complete protection against oAβ-induced loss of dendritic spine density (FIG. 8G).

### Example 7—Pharmacological Agent Topoisomerase Type I Inhibitor Increased Ube3A Levels and Decreased Ephexin-5 with No Loss of Cell Viability

Cultured primary hippocampal neurons (14 DIV) were treated with 300 nM of a topoisomerase type I inhibitor, topotecan (TOP) or a vehicle control as described in Huang et al. 2012. Some cells were treated for 24 hours and some for 72 hours. All cells were harvested and whole cell lysates prepared for Western blot analysis of Ube3A and Ephixin-5.

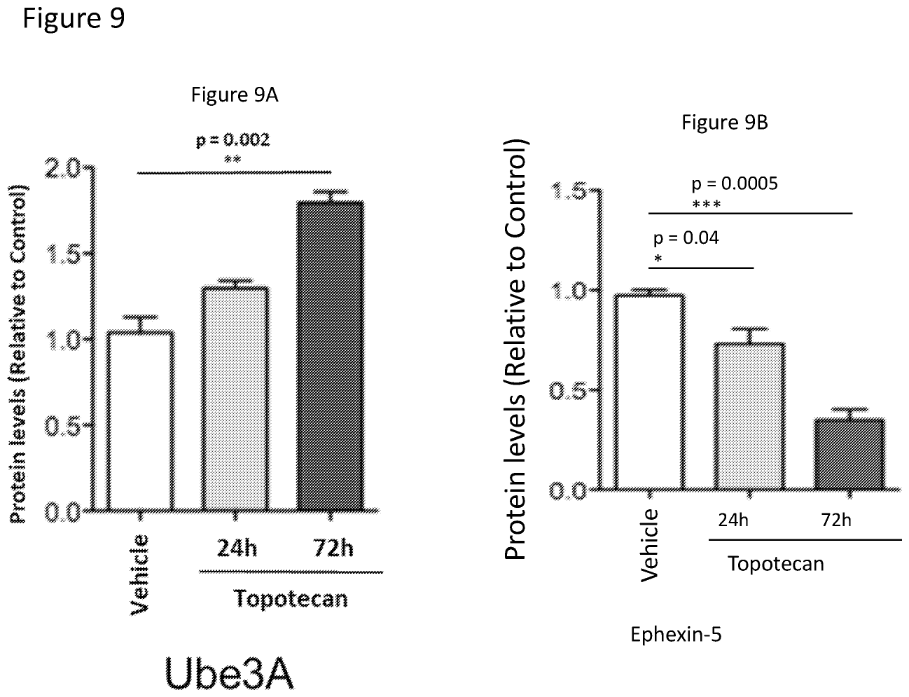

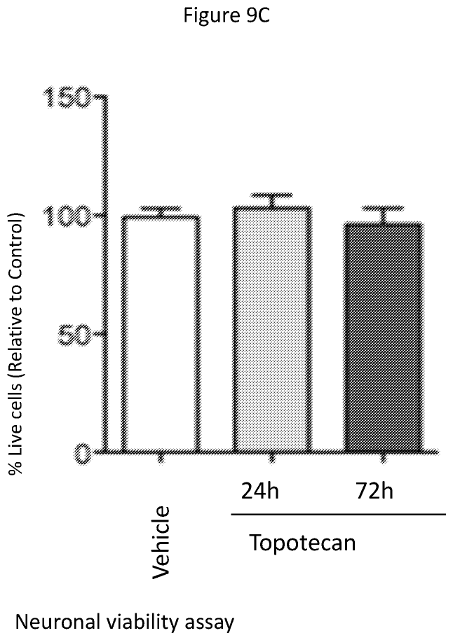

Cells were treated with TOP had Ube3A protein levels increased as early as 24 hours and persisted up to three days (FIG. 9A), correlating with a significant decrease in Ephexin-5 (FIG. 9B), and no effect on cell viability (FIG. 9C).

### Example 8—Topoisomerase Type I Inhibitor Reversed Aβ-Induced RhoA Activation

To test whether increasing Ube3A could rescue the effects of oAβ, cultured primary hippocampal neurons (14 DIV) were pretreated with 300 nM of TOP or control PBS. At day 3 the cells were treated with oligomeric Aβ for 6 hours and whole cell lysates prepared for Western blot analysis of Ube3A protein levels and levels of active GTPase levels including RhoA-GTP, Rac1-GTP and Cdc42-GTP.

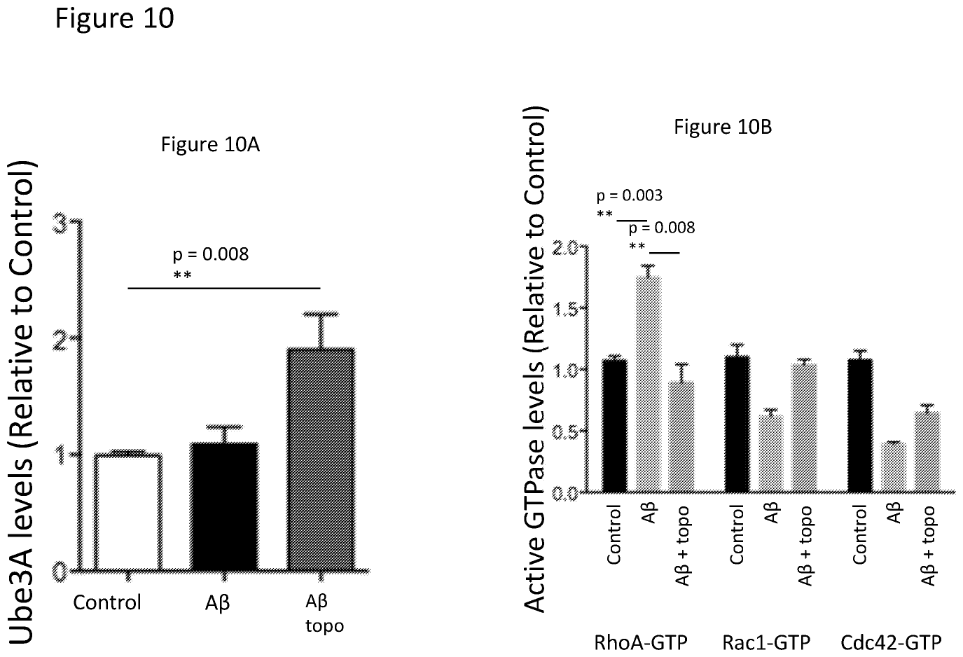

Cells treated with TOP had increased Ube3A protein levels (FIG. 10A). Consistent with the results in Example 5, cells treated with TOP also had decreased RhoA activity (FIG. 10B).

### Example 9—Topoisomerase Type I Inhibitor Reversed Aβ-Induced Spine Loss

To further test whether increasing Ube3A could rescue the effects of oAβ, cultured primary hippocampal neurons (14 DIV) were pretreated with 300 nM of TOP or control PBS. The neurons were then treated with oAβ for 24 hours, and whole cell lysates prepared for Western blot analysis, or fixed for microscopy.

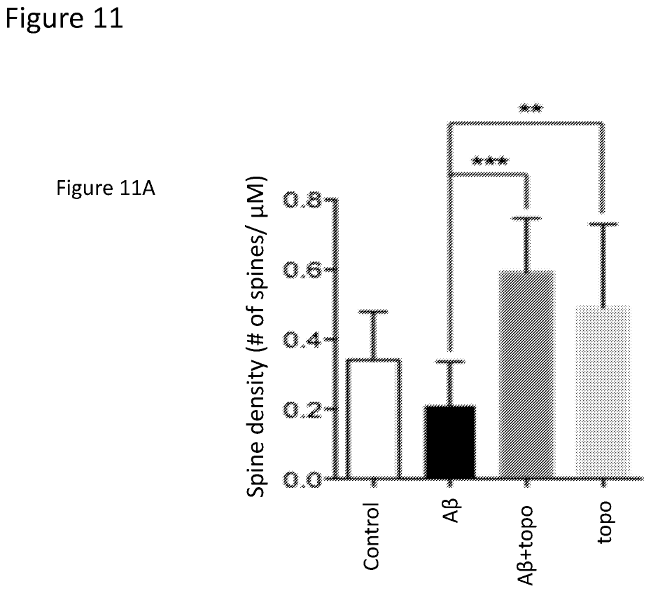

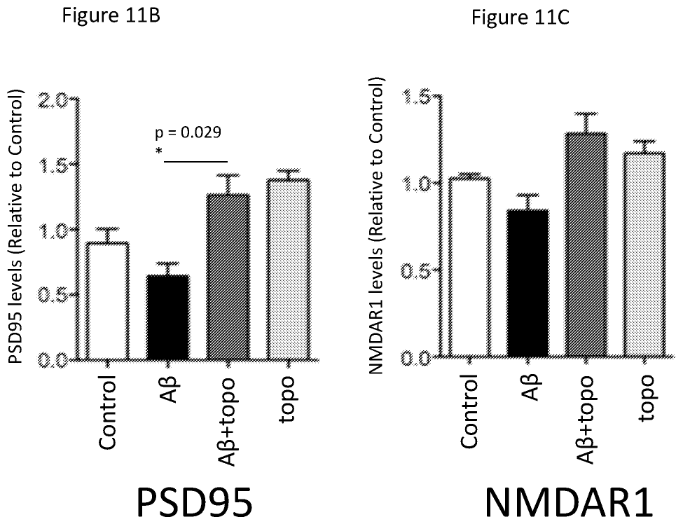

In agreement with the results of Example 6, dendritic spine analysis in fixed neurons revealed treatment with TOP resulted in a complete protection against oAβ-induced loss of dendritic spine density (FIG. 11A).

Cells were also assessed for localization of PSD95 and NMDAR1, markers of postsynaptic terminals. Cells treated with TOP had increased levels of PSD95 and NMDAR1 (FIGS. 11B and 11C).

### Example 10—Topoisomerase Type I Inhibitor Increased Learning, Memory and Dendritic Spine Density in Wild-Type Mice

Wild-type mice at two months of age were treated with a single bilateral injection of TOP (275 μg) in the hippocampus. Controls were wild type mice injected with PBS. Mice were then evaluated for working memory by Morris water maze at about 5 months, electrophysical analyses by long term potentiation recordings at about 6 months, and dendritic spine analysis at about 6.5 months.

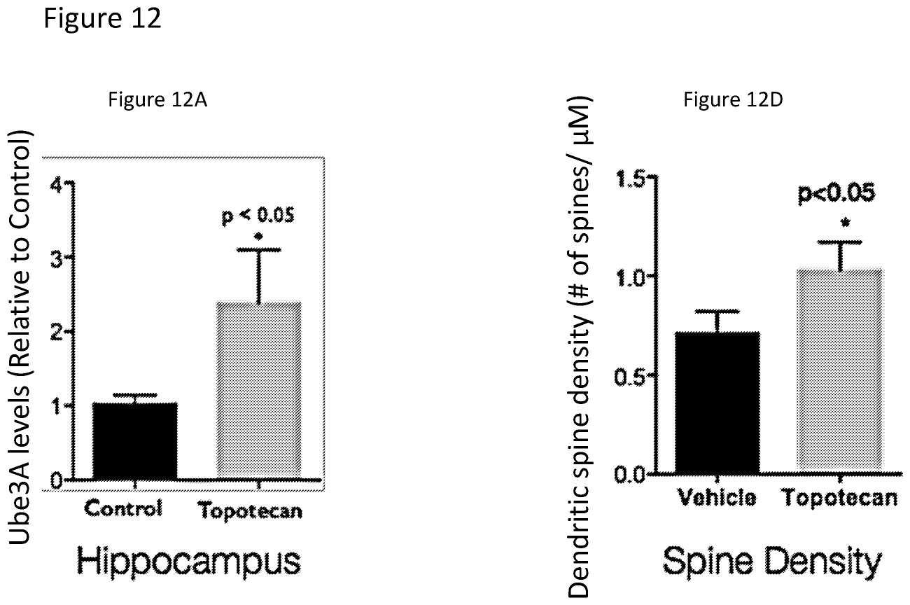

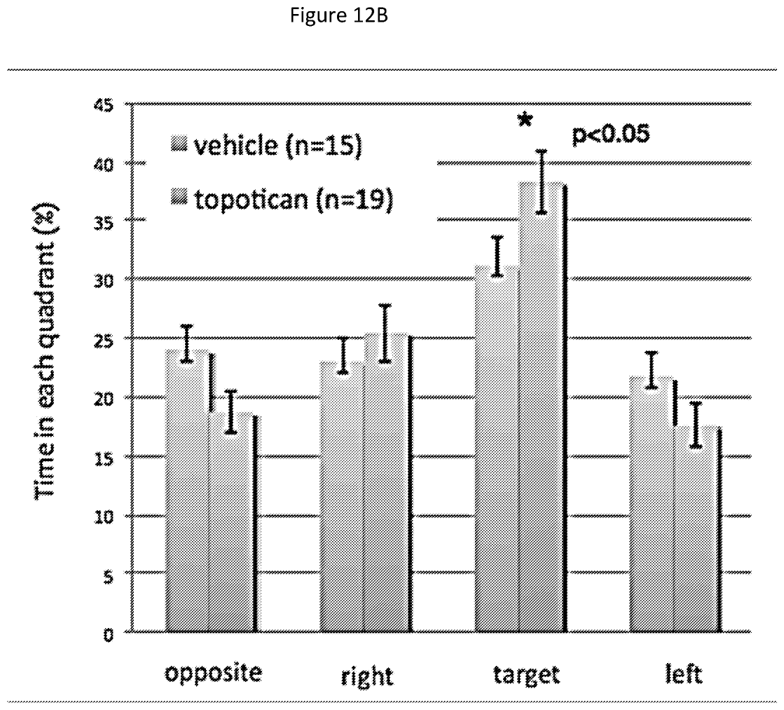

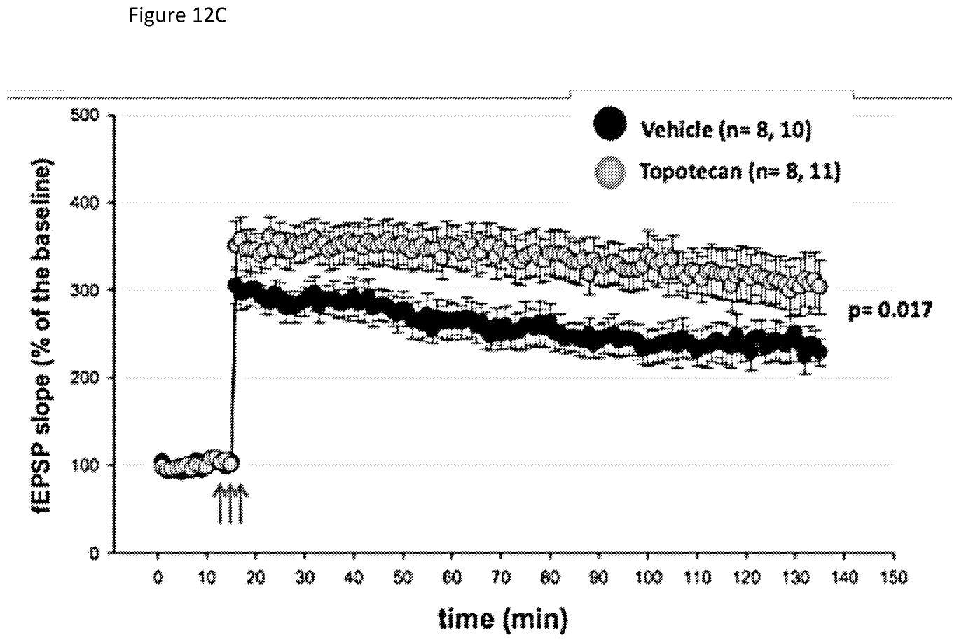

The single injection resulted in a sustained increase in Ube3A expression specifically in the hippocampus, after 1 month, and persisted up to 5 months after the single injection (FIG. 12A), suggesting a permanent unsilencing of the allele. No toxicity in these mice was observed after 9 months (data not shown).

A significant enhancement in memory was observed in TOP-injected mice after 1 month compared to PBS-injected mice, as assessed by Morris Water Maze (FIG. 12B). A similar enhancement in LTP was observed in hippocampal slices derived from TOP-treated mice (FIG. 12C). Both of these effects correlated with a significant increase in dendritic spine density (FIG. 12D).

### Example 11—Restoring Ube3A Levels by Treatment Topoisomerase Type I Inhibitor Reversed the Alzheimer's Disease Phenotype in AD Mice

To determine if TOP treatment can reverse behavioral deficits once established, J20 mice at 9-10 months of age were treated with a single bilateral injection of TOP (275 μg) in the hippocampus. Controls were wild-type mice and J20 mice injected with PBS. Mice were then evaluated for working memory by Morris water maze at about 11-12 months, electrophysical analyses by long term potentiation recordings at about 12.5 months, and dendritic spine analysis at about 12.5 months.

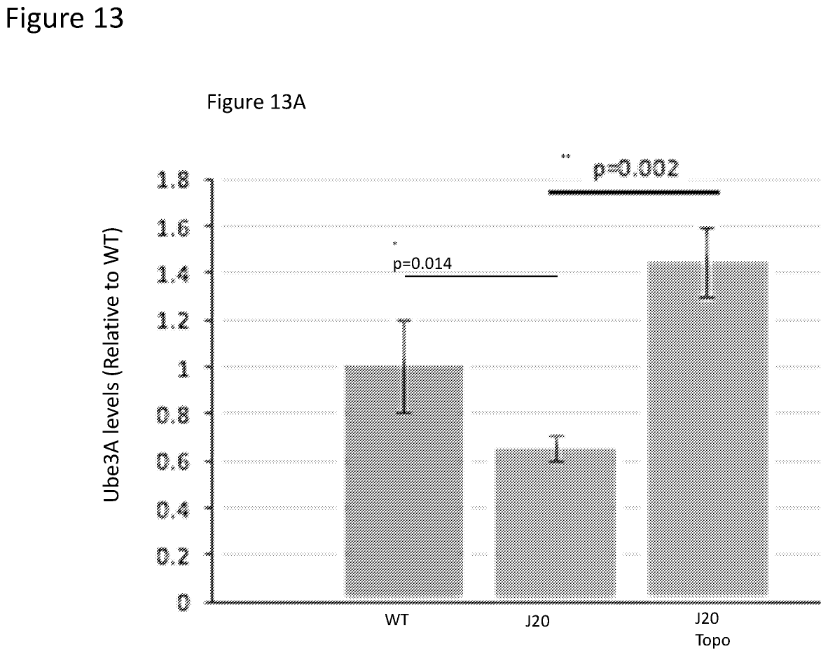

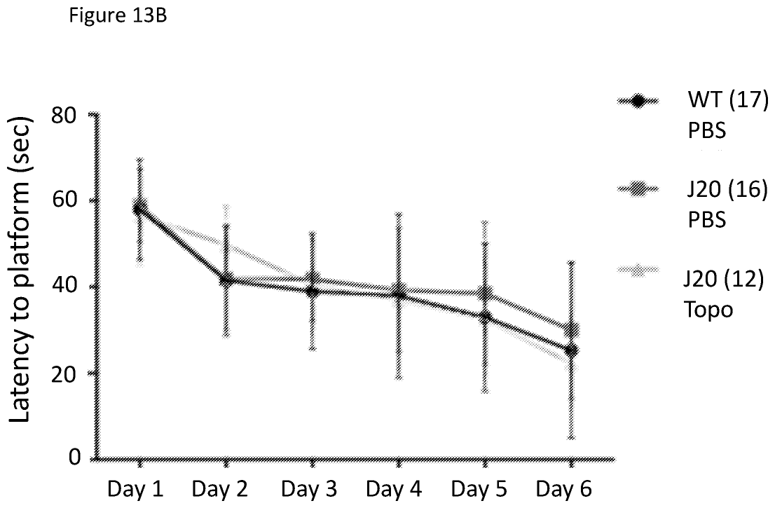

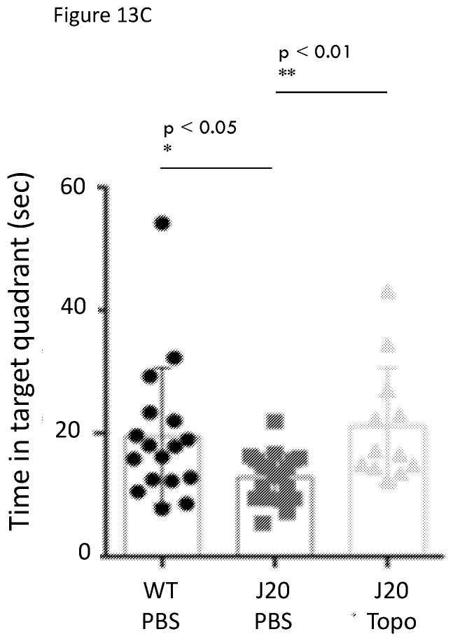

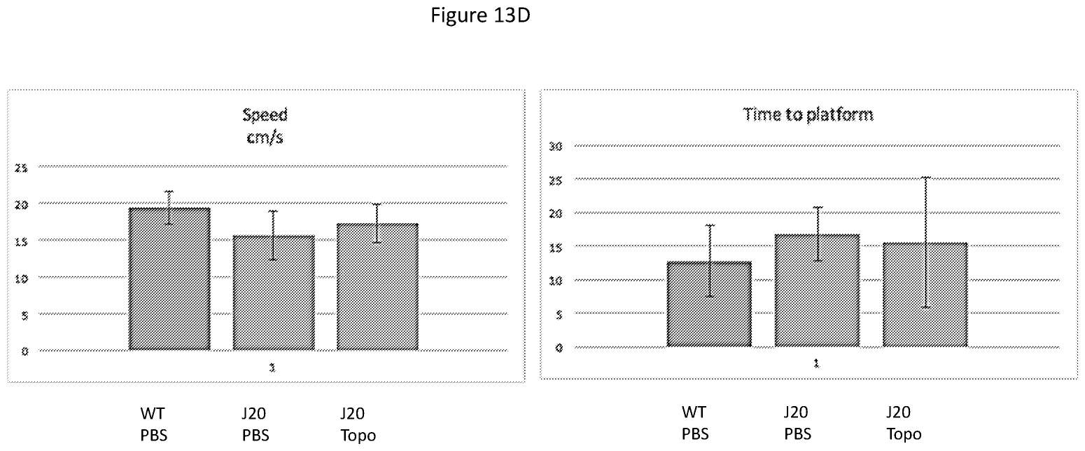

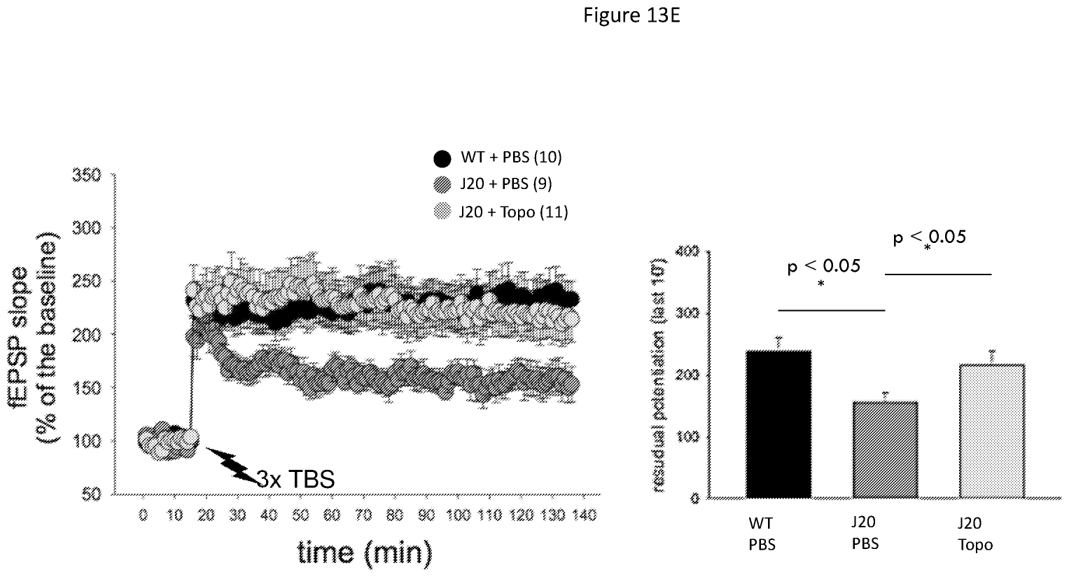

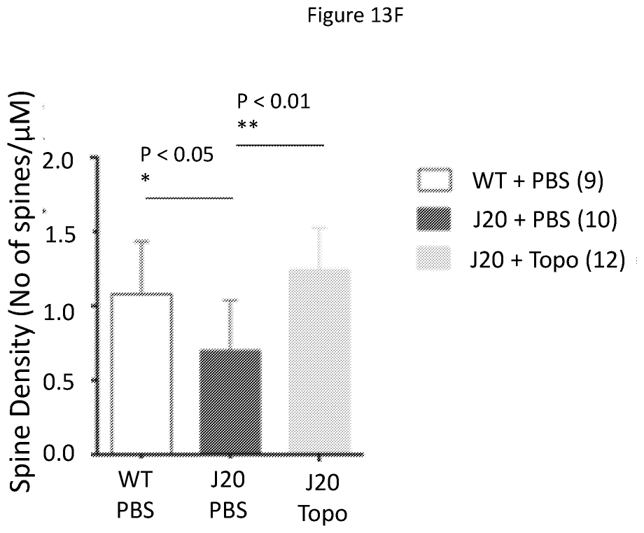

J20 mice treated with TOP had increased levels of Ube3A in the hippocampus as measured at 11-12 months (FIG. 13A).

The TOP treatment did not affect learning (FIG. 13B) but significantly increased memory (FIG. 13C). TOP treatment had no effect on motor functions or motivation (FIG. 13D). Enhancement in LTP was observed in hippocampal slices derived from TOP-treated mice as compared to PBS treated J20 mice (FIG. 13D). Both of these effects correlated with a significant increase in dendritic spine density (FIG. 13E).

### Example 12—TOP Treatment Did not Affect Aβ Load but Did not Decrease Phospho-Tau Levels

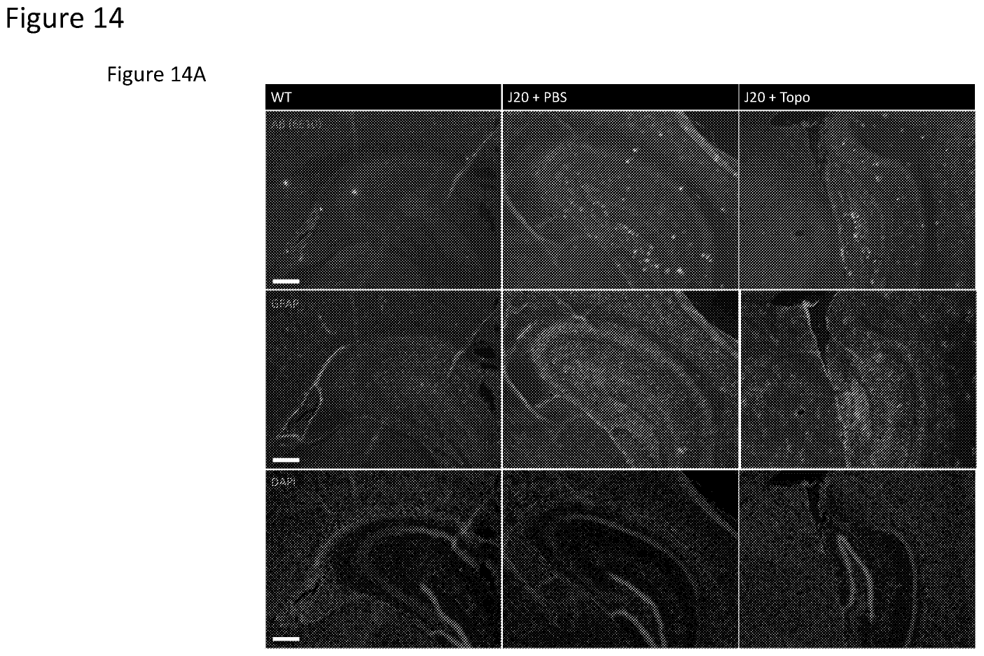

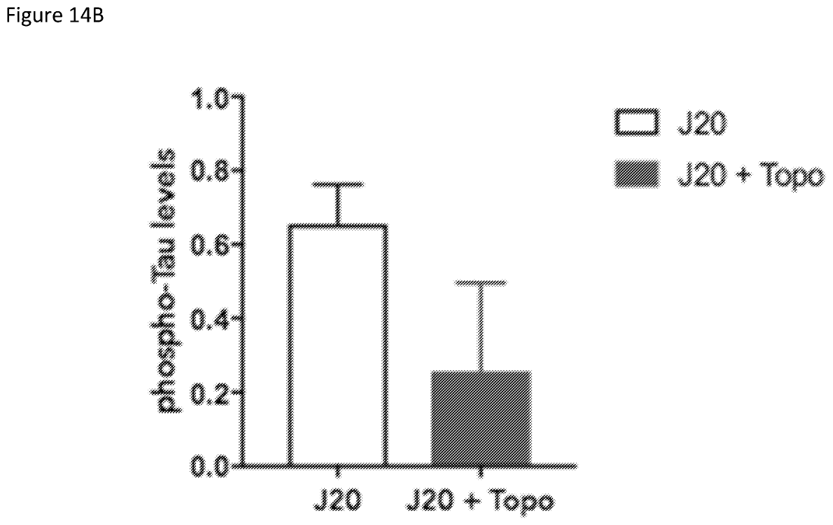

At 12.5 months of age, TOP treated J20 mice showed approximately equal accumulation of amyloid plaques as J20 mice treated with PBS and wild type mice (FIG. 14A). However, TOP treatment decreased phospho-tau levels (FIG. 14B). The relationship between Aβ and tau is unclear, however there is mounting evidence that tau and hyperphosphorylated tau may be a critical driver of the disease. The observation that topotecan also decreases phospho-tau is an important finding as it might abrogate further neurotoxic insults.

### REFERENCES

Brewer et al. Optimized survival of hippocampal neurons in B27-supplemented Neurobasal, a new serum-free medium combination. J. Neurosci. Res. 1993; 35(5):567-76.
Buchdunger et al. Inhibition of the Abl protein-tyrosine kinase in vitro and in vivo by a 2-phenylaminopyrimidine derivative. Cancer Res. 1996; 56(1):100-4.
Buiting et al. Clinical utility gene card for: Angelman Syndrome. Eur. J. Hum. Genet. 2015; 23(2).
Buiting et al. Angelman syndrome—insights into a rare neurogenetic disorder. Nat. Rev. Neurol. 2016; 12(10):584-93.
Chan et al. c-Abl phosphorylates E6AP and regulates its E3 ubiquitin ligase activity. Biochemistry. 2013; 52(18):3119-29.
Chowdhury et al. Arc/Arg3.1 interacts with the endocytic machinery to regulate AMPA receptor trafficking. Neuron. 2006; 52(3):445-59.
Cisse et al. Reversing EphB2 depletion rescues cognitive functions in Alzheimer model. Nature. 2011; 469(7328):47-52.
Davidson et al. Highly efficient small interfering RNA delivery to primary mammalian neurons induces MicroRNA-like effects before mRNA degradation. J. Neurosci. 2004; 24(45):10040-6.
Gan et al. Ballistic delivery of dyes for structural and functional studies of the nervous system. Cold Spring Harbor Protoc. 2009; 2009(4):pdb prot5202.
Gan et al. Multicolor “DiOlistic” labeling of the nervous system using lipophilic dye combinations. Neuron. 2000; 27 (2): 219-25.
Grier et al. Toward a Broader View of Ube3a in a Mouse Model of Angelman Syndrome: Expression in Brain, Spinal Cord, Sciatic Nerve and Glial Cells. PLoS One. 2015; 10(4):e0124649
Grinevich et al. Fluorescent Arc/Arg3.1 indicator mice: a versatile tool to study brain activity changes in vitro and in vivo. J. Neurosci. Methods. 2009; 184(1):25-36.
Hsiao et al. Correlative memory deficits, Abeta elevation, and amyloid plaques in transgenic mice. Science. 1996; 274(5284):99-102.
Huang et al. Topoisomerase inhibitors unsilenced the dormant allele of Ube3A in neurons Nature. 2012 481:185-89
Huesa et al. Altered distribution of RhoA in Alzheimer's disease and AbetaPP overexpressing mice. J. Alzheimers Dis. 2010; 19(1):37-56.
Huibregtse et al. A cellular protein mediates association of p53 with the E6 oncoprotein of human papillomavirus types 16 or 18. EMBO J. 1991; 10(13):4129-35.
Iwamoto et al. A general chemical method to regulate protein stability in the mammalian central nervous system. Chem. Biol. 2010; 17 (9): 981-8.
Korb and Finkbeiner. Arc in synaptic plasticity: from gene to behavior. Trends Neurosci. 2011; 34(11):591-8.
Kuhnle et al. Role of the ubiquitin ligase E6AP/UBE3A in controlling levels of the synaptic protein Arc. Proc. Natl. Acad. Sci. USA. 2013; 110(22):8888-93.
Kumar et al. Identification of HHR23A 727 as a substrate for E6-associated protein-mediated ubiquitination. J. Biol. Chem. 1999; 274(26): 18785-92.
Lacor et al. Synaptic targeting by Alzheimer's-related amyloid beta oligomers. J. Neurosci. 658 2004; 24(45): 10191-200.
Lauren et al. Cellular prion protein mediates impairment of synaptic plasticity by amyloid-beta oligomers. Nature. 2009; 457(7233):1128-32.
Margolis et al. EphB-mediated degradation of the RhoA GEF Ephexin5 relieves a developmental brake on excitatory synapse formation. Cell. 2010; 143(3):442-55.
Mishra et al. UBE3A/E6-AP regulates cell proliferation by promoting proteasomal degradation of p27. Neurobiol. Dis. 2009; 36(1):26-34.
Mucke et al. High level neuronal expression of abeta 1-42 in wild-type human amyloid protein precursor transgenic mice: synaptotoxicity without plaque formation. J. Neurosci. 2000; 20(11):4050-8.
Oda et al. Regulation of the Src family tyrosine kinase Blk through E6AP-mediated ubiquitination. Proc. Natl. Acad. Sci. USA. 1999; 96(17):9557-62.
Perez-Cruz et al. Reduced spine density in specific regions of CA1 pyramidal neurons in two transgenic mouse models of Alzheimer's disease. J. Neurosci. 2011; 31(10):3926-34
Pertz. Spatio-temporal Rho GTPase signaling—where are we now? J. Cell Sci. 2010; 123(Pt 11):1841-50.
Pozueta et al. Caspase-2 is required for dendritic spine and behavioural alterations in J20 APP transgenic mice. Nature communications. 2013; 4:1939.
Reiter et al. Expression of the Rho-GEF Pbl/ECT2 is regulated by the UBE3A E3 ubiquitin ligase. Hum. Mol. Genet. 2006; 15(18):2825-35.
Ren et al. Cloning and characterization of GEF-H1, a microtubule-associated guanine nucleotide exchange factor for Rac and Rho GTPases. J. Biol. Chem. 1998; 273(52):34954-60.
Runte et al. The IC-SNURF-SNRPN transcript serves as a host for multiple small nucleolar RNA species and as an antisense RNA for UBE3A. Hum. Mol. Genet. 2001; 10(23):2687-700.
Schaefer et al. Toward understanding RhoGTPase specificity: structure, function and local activation. Small GTPases. 2014; 5 (2): 6.
Scheff et al. Quantitative assessment of cortical synaptic density in Alzheimer's disease. Neurobiol. Aging. 1990; 11(1):29-37.
Scheff et al. Quantitative assessment of synaptic density in the entorhinal cortex in Alzheimer's disease. Ann. Neurol. 1993; 34(3):356-61.
Scheff et al. Quantitative assessment of synaptic density in the outer molecular layer of the hippocampal dentate gyrus in Alzheimer's disease. Dementia. 1996; 7(4):226-32.
Schindler et al. Structural mechanism for STI-571 inhibition of abelson tyrosine kinase. Science. 2000; 289(5486):1938-42.
Sell et al. Reducing expression of synapse restricting protein Ephexin5 ameliorates Alzheimer's-like impairment in mice. J. Clin. Invest. 2017.
Shamah et al. EphA receptors regulate growth cone dynamics through the novel guanine nucleotide exchange factor ephexin. Cell. 2001; 105(2):233
Tai et al. Destabilizing domains mediate reversible transgene expression in the brain. PLoS One. 2012; 7(9):e46269.
Tashiro and Yuste. Role of Rho GTPases in the morphogenesis and motility of dendritic spines. Methods in Enzymology. 2008; 439:285-302.
Vargas et al. EphA4 activation of c-Abl mediates synaptic loss and LTP blockade caused by amyloid beta oligomers. PLoS One. 2014; 9(3):e92309.
Woolfrey and Srivastava. Control of Dendritic Spine Morphological and Functional Plasticity by Small GTPases. Neural Plast. 2016; 2016:3025948.
Wu et al. Arc/Arg3.1 regulates an endosomal pathway essential for activity-dependent beta-amyloid generation. Cell. 2011; 147 (3): 615-28.
Yi et al. The autism-linked UBE3A T485A mutant E3 ubiquitin ligase activates the Wnt/beta catenin pathway by inhibiting the proteasome. J Biol Chem. 2017; 292(30):12503.

1. A method of treating or preventing a disease or condition characterized by synaptic dysfunction comprising administering to a subject in need thereof a therapeutically effective amount of an agent which increases the level of protein ubiquitin ligase Ube3A.

2. The method of claim 1, wherein the disease or condition is chosen from the group consisting of Alzheimer's disease, Parkinson's disease, Huntington's disease, and epilepsy.

3. The method of claim 1, wherein the subject is human.

4. The method of claim 1, wherein the level of Ube3A is increased in the brain.

5. The method of claim 1, wherein the agent is a topoisomerase type I inhibitor.

6. The method of claim 5, wherein the topoisomerase type I inhibitor is chosen from the group consisting of topotecan and irinotecan.

7. The method of claim 1, wherein the agent is administered to the subject by oral administration, parental administration, intrathecal administration or nasal administration.

8. The method of claim 6, wherein the agent is topotecan and the therapeutically effective amount ranges from about 0.75 to about 1.5 mg/ml daily.

9. The method of claim 5, wherein the agent is topotecan and a therapeutically effective amount is administered once to the subject.

10. The method of claim 6, wherein the agent is irinotecan and a therapeutically effective amount ranges from about 125 mg/ml to about 350 mg/ml.

11. The method of claim 1, wherein the agent further comprises a pharmaceutically acceptable carrier.

12. The method of claim 1, wherein the agent is a nucleotide encoding Ube3A or a variant, mutant, fragment, homologue, or derivative thereof.

13. The method of claim 12, wherein the agent further comprises a ligand, conjugate, vector, lipid, carrier, adjuvant or diluent.

14. The method of claim 1, wherein the agent is the protein ubiquitin ligase Ube3A.

15. The method of claim 14, wherein the agent further comprises a ligand, conjugate, lipid, vector, carrier, adjuvant or diluent.

16. A method of treating or preventing a disease or condition characterized by neurodegeneration comprising administering to a subject in need thereof a therapeutically effective amount of an agent which increases the level of protein ubiquitin ligase Ube3A.

17. The method of claim 16 wherein the subject is human.

18. The method of claim 16 wherein the level of Ube3A is increased in the brain.

19. The method of claim 16 wherein the agent is a topoisomerase type I inhibitor.

20. The method of claim 19, wherein the topoisomerase type I inhibitor is chosen from the group consisting of topotecan and irinotecan.

21. The method of claim 16, wherein the agent is administered to the subject by oral administration, parental administration, intrathecal administration or nasal administration.

22. The method of claim 20, wherein the agent is topotecan and the therapeutically effective amount ranges from about 0.75 to about 1.5 mg/ml daily.

23. The method of claim 20, wherein the agent is topotecan and a therapeutically effective amount is administered once to the subject.

24. The method of claim 20, wherein the agent is irinotecan and a therapeutically effective amount ranges from about 125 mg/ml to about 350 mg/ml.

25. The method of claim 16, wherein the agent further comprises a pharmaceutically acceptable carrier.

26. The method of claim 16, wherein the agent is a nucleotide encoding Ube3A or a variant, mutant, fragment, homologue, or derivative thereof.

27. The method of claim 26, wherein the agent further comprises a ligand, conjugate, vector, lipid, carrier, adjuvant or diluent.

28. The method of claim 16, wherein the agent is the protein ubiquitin ligase Ube3A.

29. The method of claim 28, wherein the agent further comprises a ligand, conjugate, lipid, vector, carrier, adjuvant or diluent.

30. A kit, comprising an agent which increases the level of protein ubiquitin ligase Ube3A and instructions as to dosage, dosing schedule, and route of administration of the agent for the treatment or prevention of a disease or condition characterized by synaptic dysfunction or neurodegeneration.

31. The kit of claim 30, wherein the agent is topoisomerase type I inhibitor is chosen from the group consisting of topotecan and irinotecan.


SEQUENCE LISTING (4 sequences)

SEQ ID NO: 1 - RNA, 21 nt, Artificial Sequence
  synthetic nucleotide
guccuuucuccguguauguuu

SEQ ID NO: 2 - RNA, 21 nt, Artificial Sequence
  synthetic nucleotide
acauacacggagaaaggacuu

SEQ ID NO: 3 - DNA, 20 bp, Artificial Sequence
  synthetic primer
cgaggacagatcaccaggag

SEQ ID NO: 4 - DNA, 20 bp, Artificial Sequence
  synthetic primer
tcattcgtgcaggcctcatt